(19) United States
(12) Patent Application Publication
(10) Pub. No.: US 2017/0224835 A1
(43) Pub. Date: Aug. 10, 2017

(54) Antibody Drug Conjugates

(71) Applicant: Sorrento Therapeutics, Inc., San Diego, CA (US)

(72) Inventors: Zhenwei Miao, San Diego, CA (US); Gang Chen, San Diego, CA (US); Tong Zhu, San Diego, CA (US); Alisher B. Khasanov, San Diego, CA (US); Dylan Deng, San Diego, CA (US); Hong Zhang, San Diego, CA (US); Zheng Yan, San Diego, CA (US)

(73) Assignee: Sorrento Therapeutics, Inc., San Diego, CA (US)

(21) Appl. No.: 15/017,174

(22) Filed: Feb. 5, 2016

Related U.S. Application Data
(60) Provisional application No. 62/113,334, filed on Feb. 6, 2015.

(51) Int. Cl. A61K 47/48; A61K 31/65

(52) U.S. Cl. CPC A61K 47/48384; A61K 31/65; A61K 47/48561

(57) ABSTRACT

There is disclosed antibody drug conjugates having anthracycline derivative drug moieties that provide improved safety and cell killing efficacy, wherein the anthracycline derivative drug moieties substitute an hydroxymethyl ketone moiety for an hydrazide or hydroxamate moiety. The disclosed cytotoxic agents (i.e., drug moieties) are conjugated to an antibody via either a Cys or a Lys residue. For Lys conjugation, the DAR (drug antibody ratio) of the majority of the ADC is 2 whereas the DAR of the majority of ADC is 4 when conjugation occurs on a Cys residue.

### CROSS REFERENCE TO RELATED APPLICATION

This patent application claims priority to pending U.S. Provisional Patent Application 62/113,334 filed 6 Feb. 2015.

### TECHNICAL FIELD

The present disclosure provides anthracycline derivative active agent (drug moiety) antibody conjugates (ADCs) that provide improved safety and cell killing efficacy by substituting a hydroxymethyl ketone moiety for a hydrazide or hydroxamate moiety on a basic anthracycline pharmacophore. The disclosed modifications provide cytotoxic agents that are conjugated to an antibody via either Cys or Lys. For Lys conjugation, the DAR (drug antibody ratio) of the majority of the ADC is 2 whereas the DAR of the majority of conjugate is 4 when conjugation occurs on Cys.

### BACKGROUND

Antibody therapy has been established for the targeted treatment of patients with cancer, immunological and angiogenic disorders (Carter (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells as well as the tumor cells sought to be eliminated (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert (2005) Current Opin. in Pharmacol. 5:543-549; Hamann (2005) Expert Opin. Ther. 15(9):1087-1103; Payne (2003) Cancer Cell 3:207-212; Trail et al (2003) Cancer Immunol. Immunother. 52:328-337; Syrigos and Epenetos (1999) Anticancer Research 19:605-614). Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (McDonagh (2006) Protein Eng. Design & Sel.; Doronina et al (2006) Bioconj. Chem. 17:114-124; Erickson et al (2006) Cancer Res. 66(8):1-8; Sanderson et al (2005) Clin. Cancer Res. 11:843-852; Jeffrey et al (2005) J. Med. Chem. 48:1344-1358; Hamblett et al (2004) Clin. Cancer Res. 10:7063-7070). Drug moieties may impart their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

The anthracycline analog, doxorubicin (ADRIAMYCIN) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al. (2007) Cardiovasc. Toxicol. 7:75-79). Immunoconjugates and prodrugs of daunorubicin and doxorubicin have been prepared and studied (Kratz et al. (2006) Current Med. Chem. 13:477-523; Jeffrey et al. (2006) Bioorganic & Med. Chem. Letters 16:358-362; Torgov et al. (2005) Bioconj. Chem. 16:717-721; Nagy et al. (2000) Proc. Natl. Acad. Sci. 97:829-834; Dubowchik et al. (2002) Bioorg. & Med. Chem. Letters 12:1529-1532; King et al. (2002) J. Med. Chem. 45:4336-4343; U.S. Pat. No. 6,630,579). The antibody-drug conjugate BR96-doxorubicin reacts specifically with the tumor-associated antigen Lewis-Y (Tolcher et al. (1999) J. Clin. Oncology 17:478-484).

Nemorubicin is a semi-synthetic anthracycline derivative which shows more potent cell killing property than some commonly used anthracylcines, such as doxorubicin and idarubicin. Because of its high cytotoxicity, it is currently being evaluated clinically to treat cancer. PNU-159682, a major metabolite of Nemorubicin from liver microsome, is significantly more cytotoxic than Nemorubicin and an ideal active agent for antibody targeted cancer therapy.

Morpholino analogs of doxorubicin and daunorubicin, formed by cyclization on the glycoside amino group, have greater potency (Acton et al. (1984) J. Med. Chem. 638-645; U.S. Pat. Nos. 4,464,529; 4,672,057; and 5,304,687). Nemorubicin is a semisynthetic analog of doxorubicin with a 2-methoxymorpholino group on the glycoside amino of doxorubicin (Grandi et al. (1990) Cancer Treat. Rew. 17:133; Ripamonti et al. (1992) Brit. J. Cancer 65:703).

Nemorubicin is named as (8S,10S)-6,8,11-trihydroxy-10-((2R,4S,5S,6S)-5-hydroxy-4-((S)-2-methoxymorpholino)-6-methyltetrahydro-2H-pyran-2-yloxy)-8-(2-hydroxyacetyl)-1-methoxy-7,8,9,10-tetrahydrotetracene-5,12-dione, with CAS Reg. No. 108852-90-0, and has the structure:

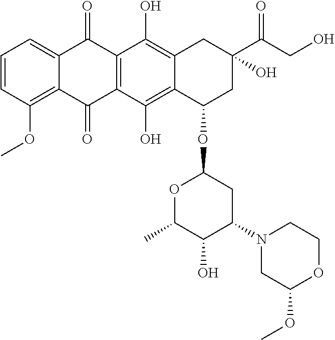 [SMILES: COc1cccc2c1C(=O)c1c(O)c3c(c(O)c1C2=O)C[C@@](O)(C(=O)CO)C[C@@H]3O[C@H]1C[C@H](N2CCO[C@H](OC)C2)[C@H](O)[C@H](C)O1]

Several metabolites of nemorubicin (MMDX) from liver microsomes have been characterized, including PNU (159682), (Quintieri et al. (2005) Clinical Cancer Research, 11(4):1608-1617; Beulz-Riche et al. (2001) Fundamental & Clinical Pharmacology, 15(6):373-378; EP 0889898; WO2004/082689; and WO2004/082579). PNU (159682) was more cytotoxic than nemorubicin and doxorubicin in vitro, and was effective in vivo tumor models. PNU (159682) is named as 3′-deamino-3″,4′-anhydro-[2″(S)-methoxy-3″(R)-oxy-4″-morpholinyl]doxorubicin, and has the structure:

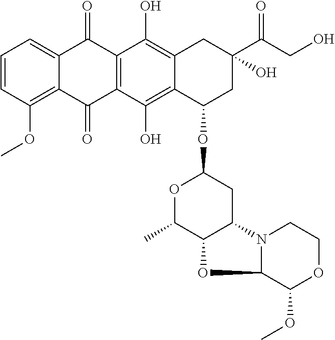 [SMILES: COc1cccc2c1C(=O)c1c(O)c3c(c(O)c1C2=O)C[C@@](O)(C(=O)CO)C[C@@H]3O[C@H]1C[C@H]2[C@H](O[C@@H]3[C@@H](OC)OCCN32)[C@H](C)O1]

Therefore there is a need in the art to further synthesize compounds in search of improved efficacy characteristics for this structure. The present disclosure provides a series of new derivative compounds showing surprisingly improved efficacy characteristics.

### SUMMARY

The present disclosure provides antibody-drug conjugates (ADCs), comprising an antibody, conjugated to a drug moiety, wherein the drug moiety is a modified tricyclic morpholino anthracycline derivative having a structure of Formula A, wherein Z is O, NH or CH₂. The drug moieties are modified with the substitution of the hydroxymethyl ketone for hydrazide or hydroxamate on the basic anthracycline pharmacophore. The disclosed modifications provide cytotoxic agents that are conjugated to an antibody via either Cys or Lys on the antibody. For Lys conjugation, the DAR (drug antibody ratio) of the majority of the conjugate is 2 whereas the DAR of the majority of conjugate is 4 when conjugation occurs on Cys.

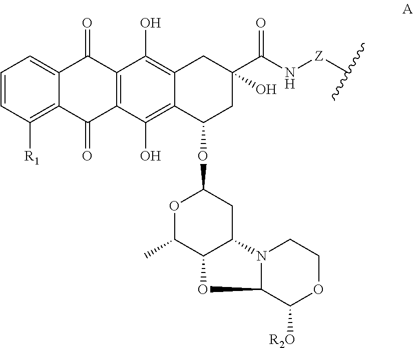

The present disclosure provides an antibody drug conjugate (ADC) having a structure of Formula I:

AbL¹-L²-D)ₙ

or a pharmaceutically acceptable salt thereof,
wherein:
Ab is an antibody;
L¹ is a connector;
L² is a linker selected from the group consisting of an amino acid, peptide, —(CH₂)ₙ—, —(CH₂CH₂O)ₙ—, p-aminobenzyl (PAB), Val-Cit-PAB, Val-Ala-PAB, Ala-Ala-Asn-PAB, and combinations thereof,
D is a drug moiety of an active agent having the structure of Formula II:

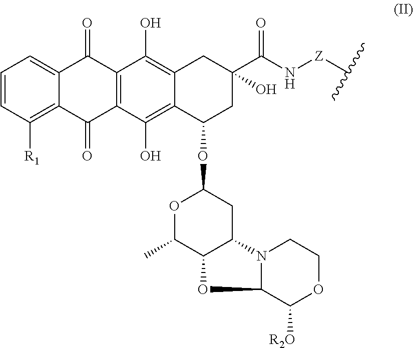

wherein Z═O, NH, or CH₂,

### R₁═H, OH, or OMe,

R₂ is a C1-C5 alkyl group, and
n is an integer from 1-10.

Preferably, for Cys conjugation, -L¹-L² is selected from the group consisting of

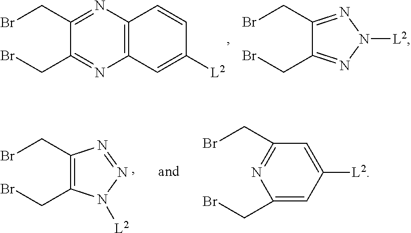

Preferably, for Lys conjugation, -L¹-L² is selected from the group consisting of

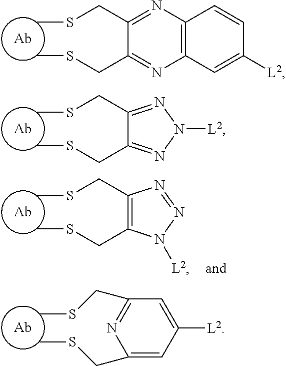

The present disclosure further provides a synthesis method for synthesizing a structure of Formula I

AbL¹-L²-D)ₙ

or a pharmaceutically acceptable salt thereof,
wherein:
Ab is an antibody
L¹ is a connector
L² is a linker selected from the group consisting of an amino acid, peptide, —(CH₂)ₙ—, —(CH₂CH₂O)ₙ—, p-aminobenzyl (PAB), Val-Cit-PAB, Val-Ala-PAB, Ala-Ala-Asn-PAB, and combinations thereof
D is a drug moiety having a structure of Formula II:

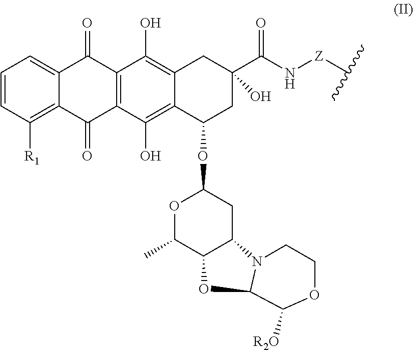

wherein Z═O, NH, or CH₂,

### R₁═H, OH, or OMe,

R₂ is a C1-C5 alkyl group, and
n is an integer from 1-10
Preferably, Ab-L¹-L² is

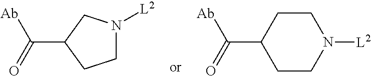

### BRIEF DESCRIPTION OF THE FIGURES

FIG. 1 shows in vivo efficacy of ADC 20 (anti-Her2 antibody) in an N87 xenograft model.

FIG. 2 shows in vivo safety of ADC 20 (anti-Her2 antibody) in N87 cells in a xenograft model.

FIG. 3 shows in vivo efficacy of ADC 35 (anti-Her2 antibody) in an N87 xenograft model.

FIG. 4 shows in vivo safety of ADC 35 (anti-Her2 antibody) in an N87 xenograft model.

### DETAILED DESCRIPTION

The present disclosure provides examples of the following disclosed antibody conjugates, listed for conjugation to a Lys on an antibody or to a Cys on an antibody.

TABLE 1
Structures of compounds synthesized (for Lys conjugation)

| Compound | |
|---|---|
| ID | Structure |
| 2 | 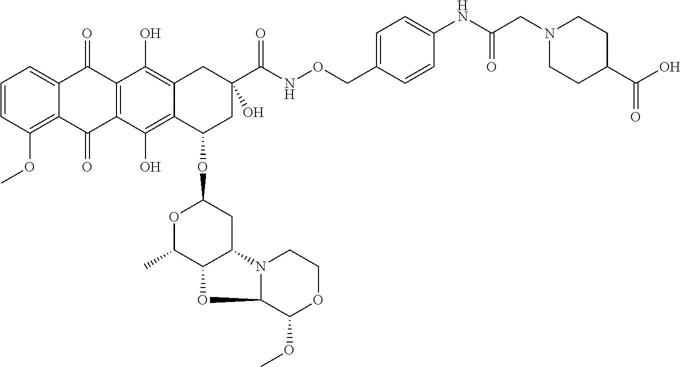 [SMILES: COc1cccc2c1C(=O)c1c(O)c3c(c(O)c1C2=O)C[C@@](O)(C(=O)NOCc1ccc(NC(=O)CN2CCC(C(=O)O)CC2)cc1)C[C@@H]3O[C@H]1C[C@H]2[C@H](O[C@@H]3[C@@H](OC)OCCN32)[C@H](C)O1] |
| 3 | 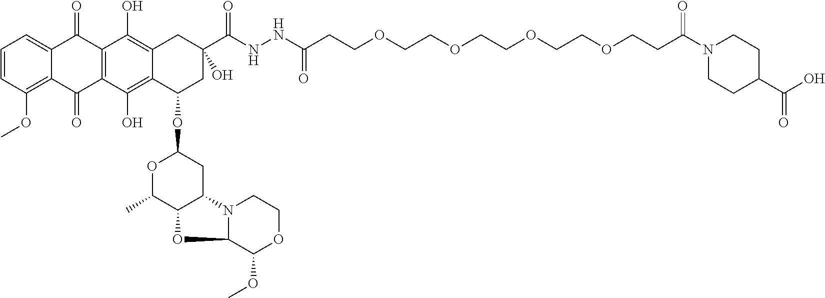 [SMILES: COc1cccc2c1C(=O)c1c(O)c3c(c(O)c1C2=O)C[C@@](O)(C(=O)NNC(=O)CCOCCOCCOCCOCCC(=O)N1CCC(C(=O)O)CC1)C[C@@H]3O[C@H]1C[C@H]2[C@H](O[C@@H]3[C@@H](OC)OCCN32)[C@H](C)O1] |
| 4 | 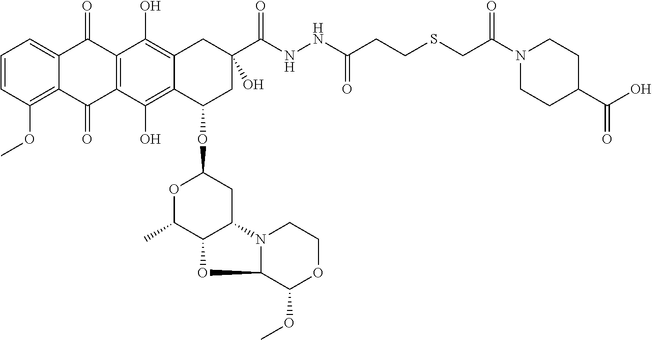 [SMILES: COc1cccc2c1C(=O)c1c(O)c3c(c(O)c1C2=O)C[C@@](O)(C(=O)NNC(=O)CCSCC(=O)N1CCC(C(=O)O)CC1)C[C@@H]3O[C@H]1C[C@H]2[C@H](O[C@@H]3[C@@H](OC)OCCN32)[C@H](C)O1] |
| 5 | 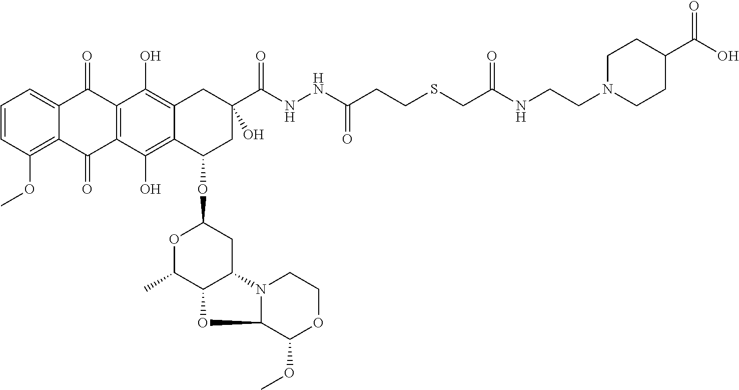 [SMILES: COc1cccc2c1C(=O)c1c(O)c3c(c(O)c1C2=O)C[C@@](O)(C(=O)NNC(=O)CCSCC(=O)NCCN1CCC(C(=O)O)CC1)C[C@@H]3O[C@H]1C[C@H]2[C@H](O[C@@H]3[C@@H](OC)OCCN32)[C@H](C)O1] |
| 6 | 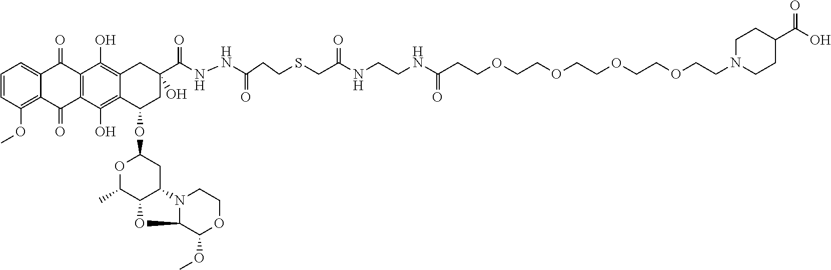 [SMILES: COc1cccc2c1C(=O)c1c(O)c3c(c(O)c1C2=O)C[C@@](O)(C(=O)NNC(=O)CCSCC(=O)NCCNC(=O)CCOCCOCCOCCOCCN1CCC(C(=O)O)CC1)C[C@@H]3O[C@H]1C[C@H]2[C@H](O[C@@H]3[C@@H](OC)OCCN32)[C@H](C)O1] |
| 7 | 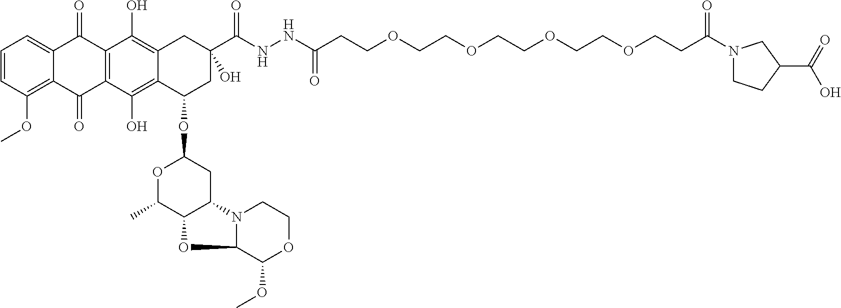 [SMILES: COc1cccc2c1C(=O)c1c(O)c3c(c(O)c1C2=O)C[C@@](O)(C(=O)NNC(=O)CCOCCOCCOCCOCCC(=O)N1CCC(C(=O)O)C1)C[C@@H]3O[C@H]1C[C@H]2[C@H](O[C@@H]3[C@@H](OC)OCCN32)[C@H](C)O1] |
| 8 | 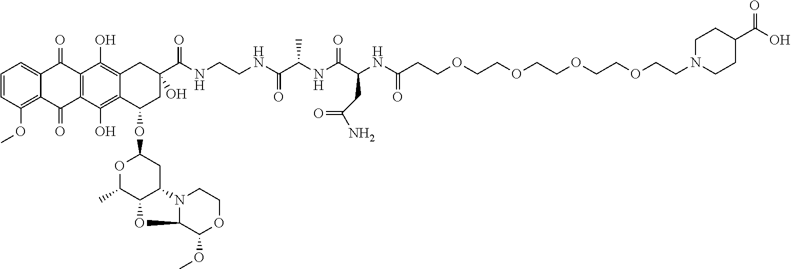 [SMILES: COc1cccc2c1C(=O)c1c(O)c3c(c(O)c1C2=O)C[C@@](O)(C(=O)NCCNC(=O)[C@H](C)NC(=O)[C@H](CC(N)=O)NC(=O)CCOCCOCCOCCOCCN1CCC(C(=O)O)CC1)C[C@@H]3O[C@H]1C[C@H]2[C@H](O[C@@H]3[C@@H](OC)OCCN32)[C@H](C)O1] |

TABLE 2
Structures of compounds synthesized (for Cys conjugation)

| Compound | |
|---|---|
| ID | Structure |
| 9 | 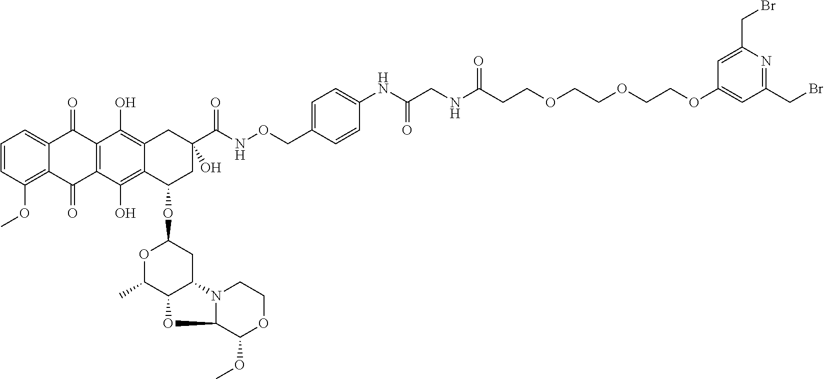 [SMILES: COc1cccc2c1C(=O)c1c(O)c3c(c(O)c1C2=O)C[C@@](O)(C(=O)NOCc1ccc(NC(=O)CNC(=O)CCOCCOCCOc2cc(CBr)nc(CBr)c2)cc1)C[C@@H]3O[C@H]1C[C@H]2[C@H](O[C@@H]3[C@@H](OC)OCCN32)[C@H](C)O1] |
| 10 | 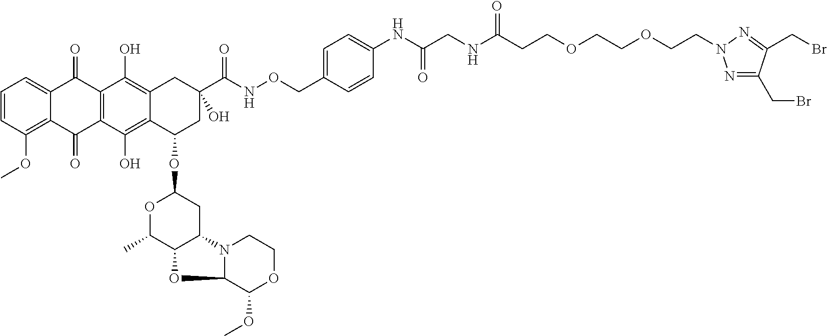 [SMILES: COc1cccc2c1C(=O)c1c(O)c3c(c(O)c1C2=O)C[C@@](O)(C(=O)NOCc1ccc(NC(=O)CNC(=O)CCOCCOCCn2nc(CBr)c(CBr)n2)cc1)C[C@@H]3O[C@H]1C[C@H]2[C@H](O[C@@H]3[C@@H](OC)OCCN32)[C@H](C)O1] |
| 11 | 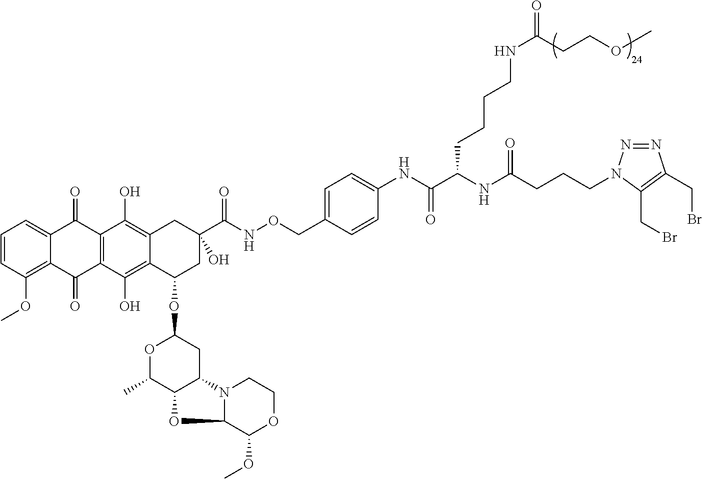 |
| 12 | 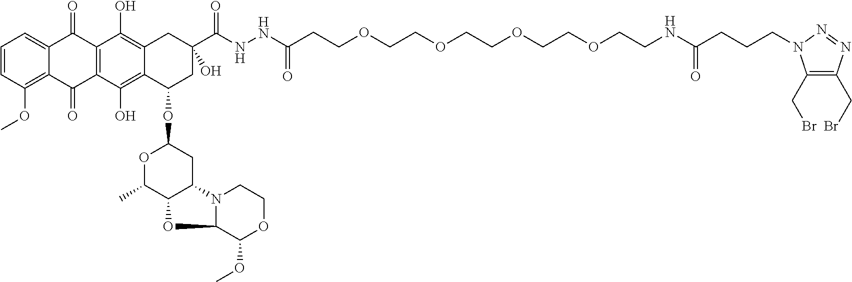 [SMILES: COc1cccc2c1C(=O)c1c(O)c3c(c(O)c1C2=O)C[C@@](O)(C(=O)NNC(=O)CCOCCOCCOCCOCCNC(=O)CCCn1nnc(CBr)c1CBr)C[C@@H]3O[C@H]1C[C@H]2[C@H](O[C@@H]3[C@@H](OC)OCCN32)[C@H](C)O1] |
| 13 | 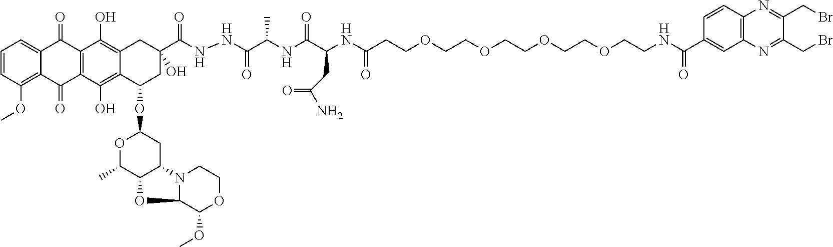 [SMILES: COc1cccc2c1C(=O)c1c(O)c3c(c(O)c1C2=O)C[C@@](O)(C(=O)NNC(=O)[C@H](C)NC(=O)[C@H](CC(N)=O)NC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1] |
| 14 | 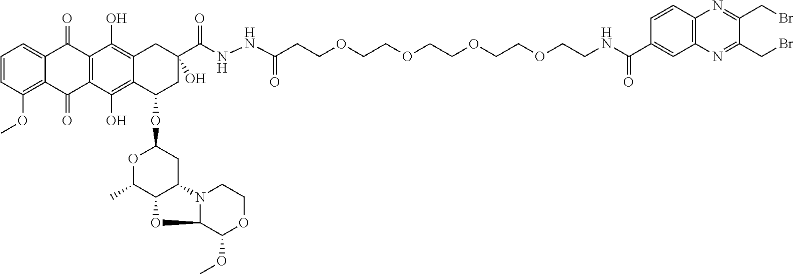 [SMILES: COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1] |
| 15 | 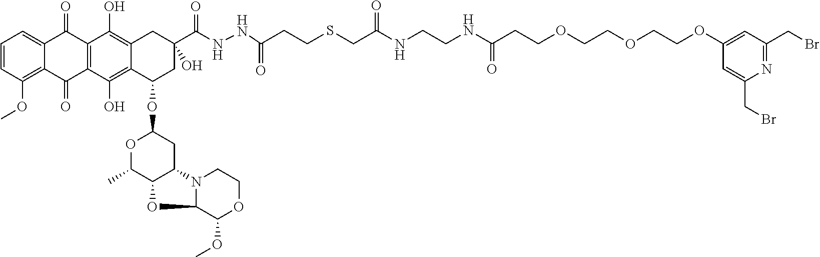 [SMILES: COc1cccc2c1C(=O)c1c(O)c3c(c(O)c1C2=O)C[C@@](O)(C(=O)NNC(=O)CCSCC(=O)NCCNC(=O)CCOCCOCCOc1cc(CBr)nc(CBr)c1)C[C@@H]3O[C@H]1C[C@H]2[C@H](O[C@@H]3[C@@H](OC)OCCN32)[C@H](C)O1] |
| 16 | 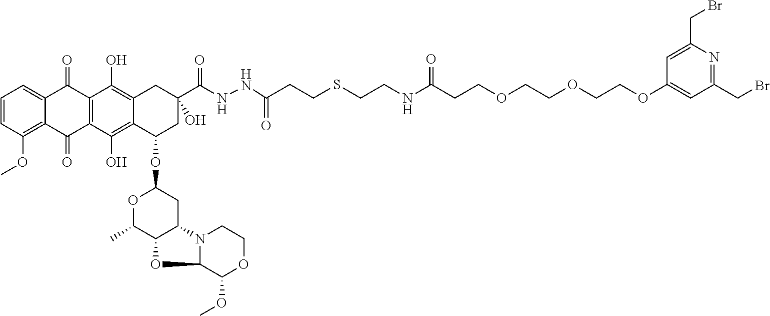 [SMILES: COc1cccc2c1C(=O)c1c(O)c3c(c(O)c1C2=O)C[C@@](O)(C(=O)NNC(=O)CCSCCNC(=O)CCOCCOCCOc1cc(CBr)nc(CBr)c1)C[C@@H]3O[C@H]1C[C@H]2[C@H](O[C@@H]3[C@@H](OC)OCCN32)[C@H](C)O1] |
| 17 | 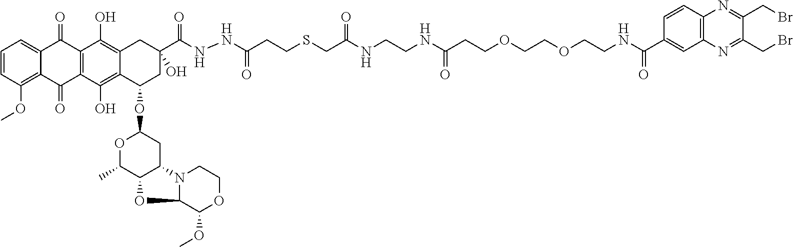 [SMILES: COc1cccc2c1C(=O)c1c(O)c3c(c(O)c1C2=O)C[C@@](O)(C(=O)NNC(=O)CCSCC(=O)NCCNC(=O)CCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1] |
| 18 | 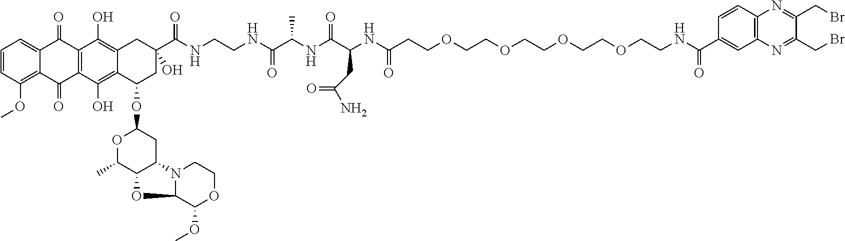 [SMILES: COc1cccc2c1C(=O)c1c(O)c3c(c(O)c1C2=O)C[C@@](O)(C(=O)NCCNC(=O)[C@H](C)NC(=O)[C@H](CC(N)=O)NC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1] |

TABLE 3
Structures of antibody-drug conjugates synthesized

| Conjugate | |
|---|---|
| ID | Structure |
| 19 | 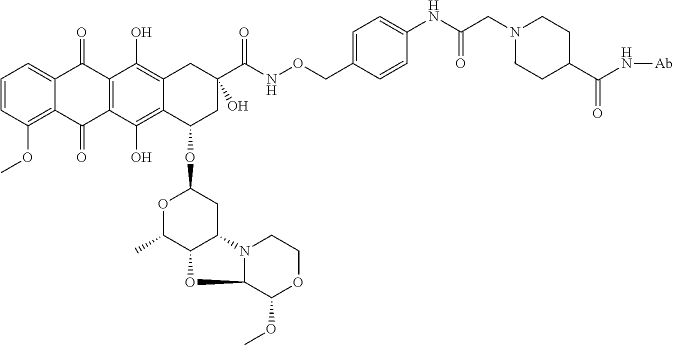 |
| 20 | 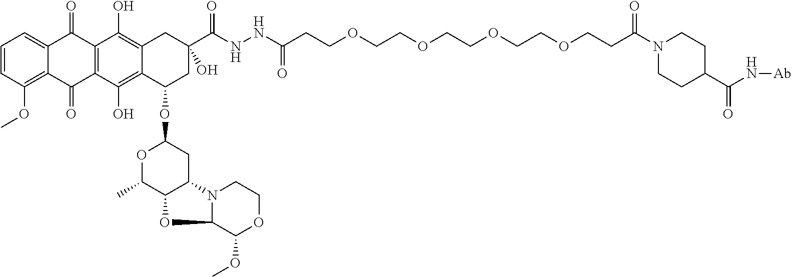 |
| 21 | 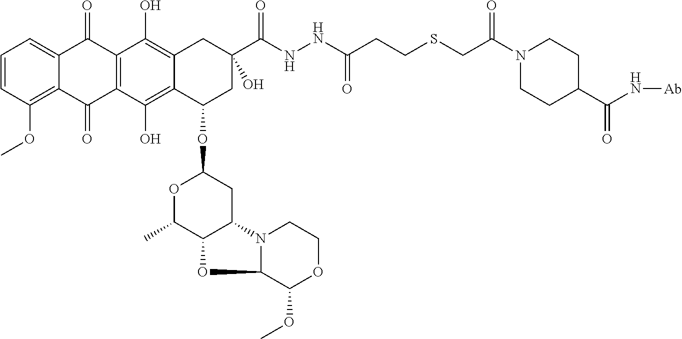 |
| 22 | 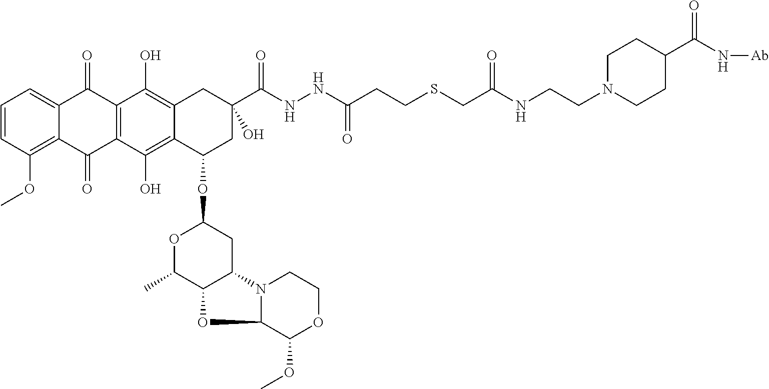 |
| 23 | 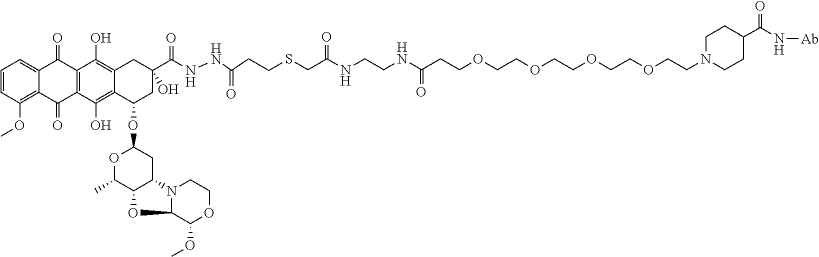 |
| 24 | 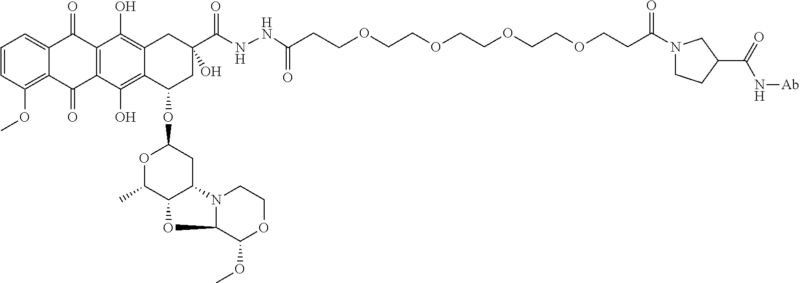 |
| 25 | 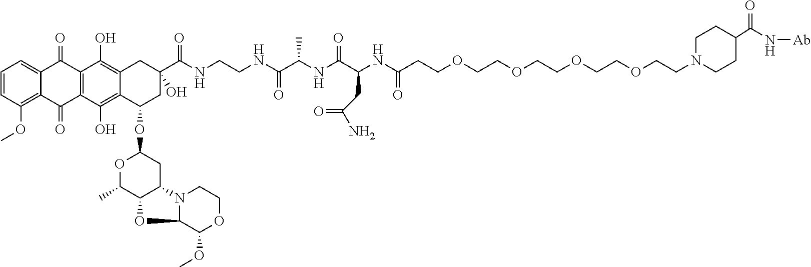 |
| 26 | 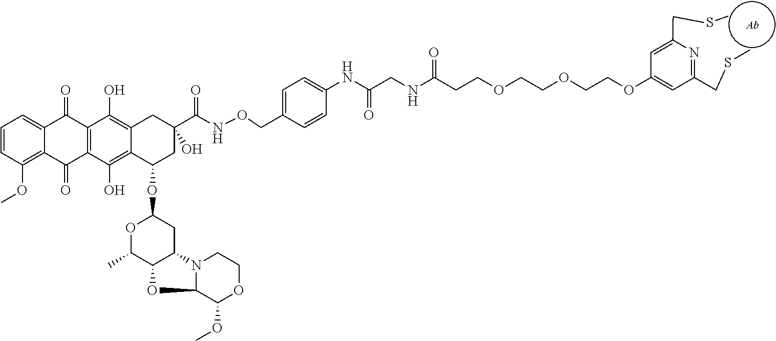 [SMILES: COc1cccc2c1C(=O)c1c(O)c3c(c(O)c1C2=O)C[C@@](O)(C(=O)NOCc1ccc(NC(=O)CNC(=O)CCOCCOCCOc2cc(CSC)nc(CSC)c2)cc1)C[C@@H]3O[C@H]1C[C@H]2[C@H](O[C@@H]3[C@@H](OC)OCCN32)[C@H](C)O1] |
| 27 | 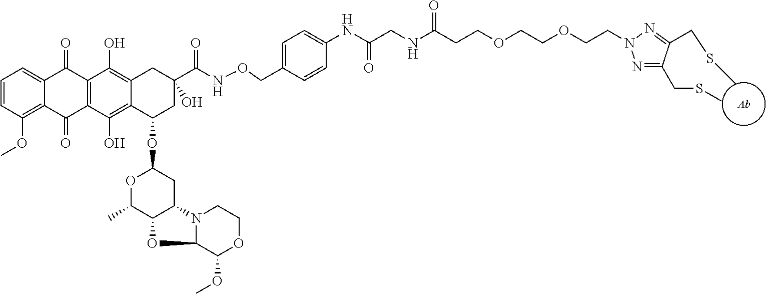 [SMILES: COc1cccc2c1C(=O)c1c(O)c3c(c(O)c1C2=O)C[C@@](O)(C(=O)NOCc1ccc(NC(=O)CNC(=O)CCOCCOCCn2nc(CSC)c(CSC)n2)cc1)C[C@@H]3O[C@H]1C[C@H]2[C@H](O[C@@H]3[C@@H](OC)OCCN32)[C@H](C)O1] |
| 28 | 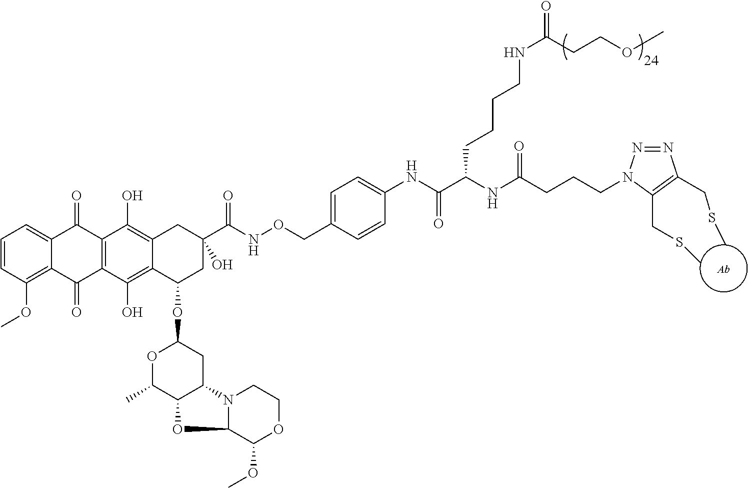 |
| 29 | 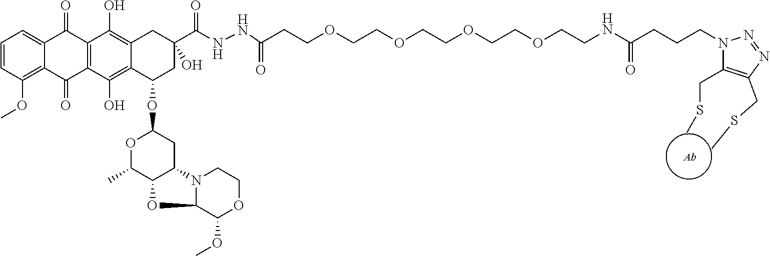 [SMILES: COc1cccc2c1C(=O)c1c(O)c3c(c(O)c1C2=O)C[C@@](O)(C(=O)NNC(=O)CCOCCOCCOCCOCCNC(=O)CCCn1nnc(CSC)c1CSC)C[C@@H]3O[C@H]1C[C@H]2[C@H](O[C@@H]3[C@@H](OC)OCCN32)[C@H](C)O1] |
| 30 | 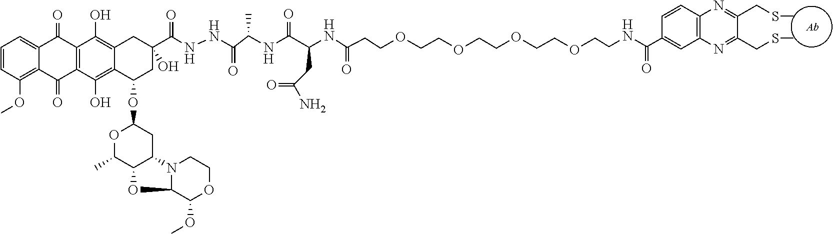 [SMILES: COc1cccc2c1C(=O)c1c(O)c3c(c(O)c1C2=O)C[C@@](O)(C(=O)NNC(=O)[C@H](C)NC(=O)[C@H](CC(N)=O)NC(=O)CCOCCOCCOCCOCCNC(=O)c1ccc2nc(CSC)c(CSC)nc2c1)C[C@@H]3O[C@H]1C[C@H]2[C@H](O[C@@H]3[C@@H](OC)OCCN32)[C@H](C)O1] |
| 31 | 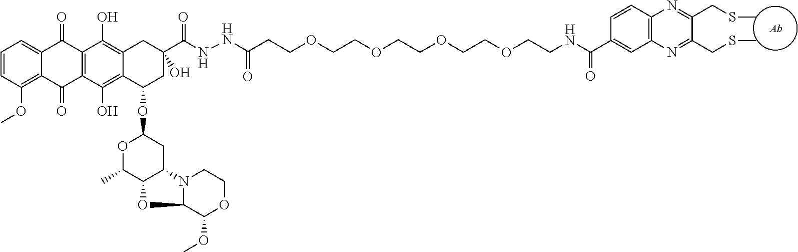 [SMILES: COc1cccc2c1C(=O)c1c(O)c3c(c(O)c1C2=O)C[C@@](O)(C(=O)NNC(=O)CCOCCOCCOCCOCCNC(=O)c1ccc2nc(CSC)c(CSC)nc2c1)C[C@@H]3O[C@H]1C[C@H]2[C@H](O[C@@H]3[C@@H](OC)OCCN32)[C@H](C)O1] |
| 32 | 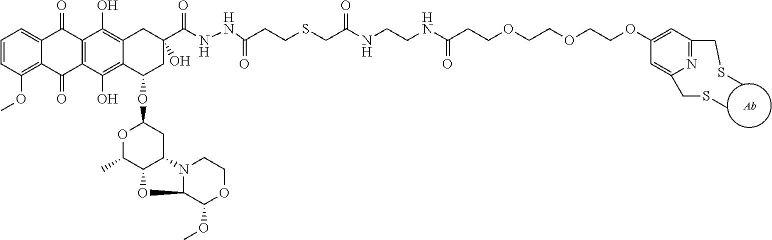 [SMILES: COc1cccc2c1C(=O)c1c(O)c3c(c(O)c1C2=O)C[C@@](O)(C(=O)NNC(=O)CCSCC(=O)NCCNC(=O)CCOCCOCCOc1cc(CSC)nc(CSC)c1)C[C@@H]3O[C@H]1C[C@H]2[C@H](O[C@@H]3[C@@H](OC)OCCN32)[C@H](C)O1] |
| 33 | 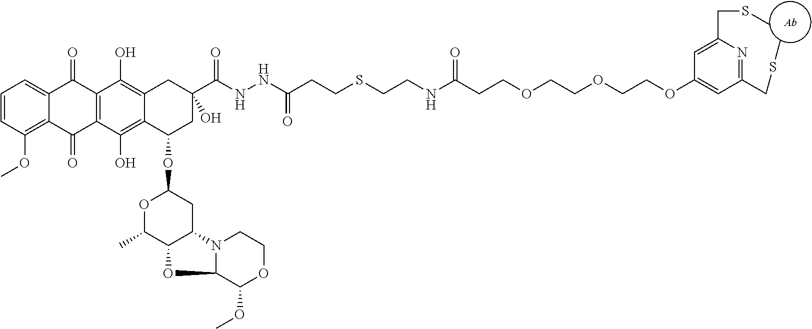 [SMILES: COc1cccc2c1C(=O)c1c(O)c3c(c(O)c1C2=O)C[C@@](O)(C(=O)NNC(=O)CCSCCNC(=O)CCOCCOCCOc1cc(CSC)nc(CSC)c1)C[C@@H]3O[C@H]1C[C@H]2[C@H](O[C@@H]3[C@@H](OC)OCCN32)[C@H](C)O1] |
| 34 | 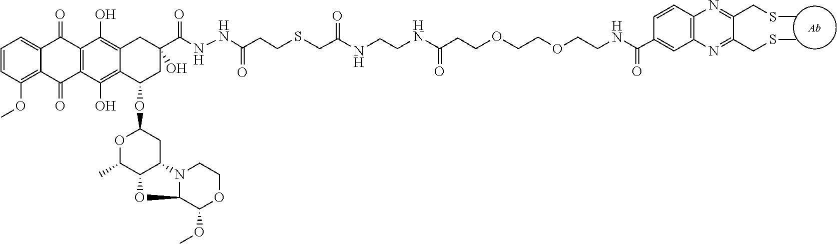 [SMILES: COc1cccc2c1C(=O)c1c(O)c3c(c(O)c1C2=O)C[C@@](O)(C(=O)NNC(=O)CCSCC(=O)NCCNC(=O)CCOCCOCCNC(=O)c1ccc2nc(CSC)c(CSC)nc2c1)C[C@@H]3O[C@H]1C[C@H]2[C@H](O[C@@H]3[C@@H](OC)OCCN32)[C@H](C)O1] |
| 35 | 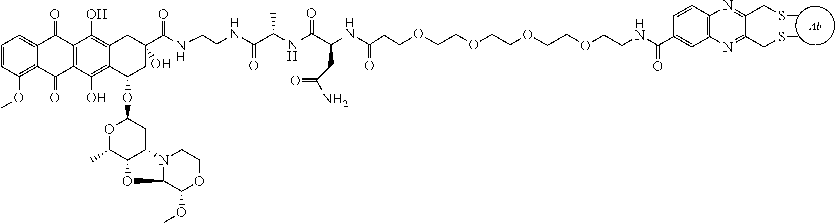 [SMILES: COc1cccc2c1C(=O)c1c(O)c3c(c(O)c1C2=O)C[C@@](O)(C(=O)NCCNC(=O)[C@H](C)NC(=O)[C@H](CC(N)=O)NC(=O)CCOCCOCCOCCOCCNC(=O)c1ccc2nc(CSC)c(CSC)nc2c1)C[C@@H]3O[C@H]1C[C@H]2[C@H](O[C@@H]3[C@@H](OC)OCCN32)[C@H](C)O1] |

Ab is preferably an IgG class antibody.

### Definitions

As used herein, common organic abbreviations are defined as follows:
Ac Acetyl
aq. Aqueous
BOC or Boc tert-Butoxycarbonyl
BrOP bromo tris(dimethylamino) phosphonium hexafluorophosphate
Bu n-Butyl
° C. Temperature in degrees Centigrade
DCM methylene chloride
DEPC Diethylcyanophosphonate
DIC diisopropylcarbodiimide
DIEA Diisopropylethylamine
DMA N,N′-Dimethylformamide
DMF N,N′-Dimethylformamide
EDC 1-Ethyl-3-(3-dimethylaminopropyl)carbodiimide
Et Ethyl
EtOAc Ethyl acetate
Eq Equivalents
Fmoc 9-Fluorenylmethoxycarbonyl
g Gram(s)
h Hour (hours)
HATU 2-(1H-7-azabenzotriazol-1-yl)-1,1,3,3-tetramethyl uronium hexafluorophosphate
HOBT N-Hydroxybenzotriazole
HOSu N-Hydroxysuccinimide
HPLC High-performance liquid chromatography
LC/MS Liquid chromatography-mass spectrometry
Me Methyl
MeOH Methanol
MeCN Acetonitrile
mL Milliliter(s)
MS mass spectrometry
PAB p-aminobenzyl
RP-HPLC reverse phase HPLC
rt room temperature
t-Bu tert-Butyl
TEA Triethylamine
Tert, t tertiary
TFA Trifluoracetic acid
THF Tetrahydrofuran
TLC Thin-layer chromatography
L Microliter(s)

### General Procedure—

Formation of an Activated Ester (e.g. NHS) from an Acid

An acid was dissolved in DCM and DMF was added to aid dissolution if necessary. N-hydroxysuccinimide (1.5 eq) was added, followed by EDC.HCl (1.5 eq). The reaction mixture was stirred at room temperature for 1 h until most of the acid was consumed. The progress of the reaction was monitored by RP-HPLC. The mixture was then diluted with DCM and washed successively with citric acid (aq. 10%) and brine. The organic layer was dried and concentrated to dryness. The crude product was optionally purified by RP-HPLC or silica gel column chromatography.

### Example 1. Preparation of Compound 2

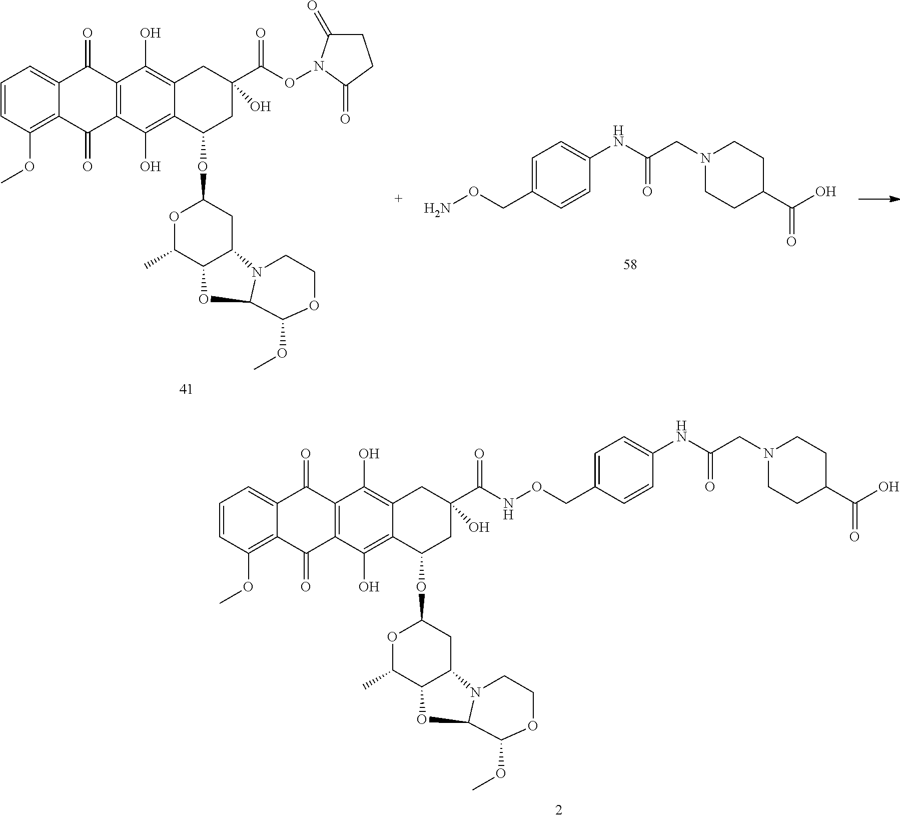 [SMILES: COc1cccc2c1C(=O)c1c(O)c3c(c(O)c1C2=O)C[C@@](O)(C(=O)ON1C(=O)CCC1=O)C[C@@H]3O[C@H]1C[C@H]2[C@H](O[C@@H]3[C@@H](OC)OCCN32)[C@H](C)O1 ; NOCc1ccc(NC(=O)CN2CCC(C(=O)O)CC2)cc1 ; COc1cccc2c1C(=O)c1c(O)c3c(c(O)c1C2=O)C[C@@](O)(C(=O)NOCc1ccc(NC(=O)CN2CCC(C(=O)O)CC2)cc1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and hydroxylamine 58 (45 mg, 0.15 mmol). The mixture was stirred at room termperature for 16 h, then diluted with DCM (30 mL). The mixture was washed with brine. The organic layer was dried and evaporated to dryness. The residue was purified by column (silica gel, DCM:MeOH, 9:1) to give compound 3 (46 mg, 50%). MS m/z 917.4 (M+H).

### Example 2. Preparation of Compound 3

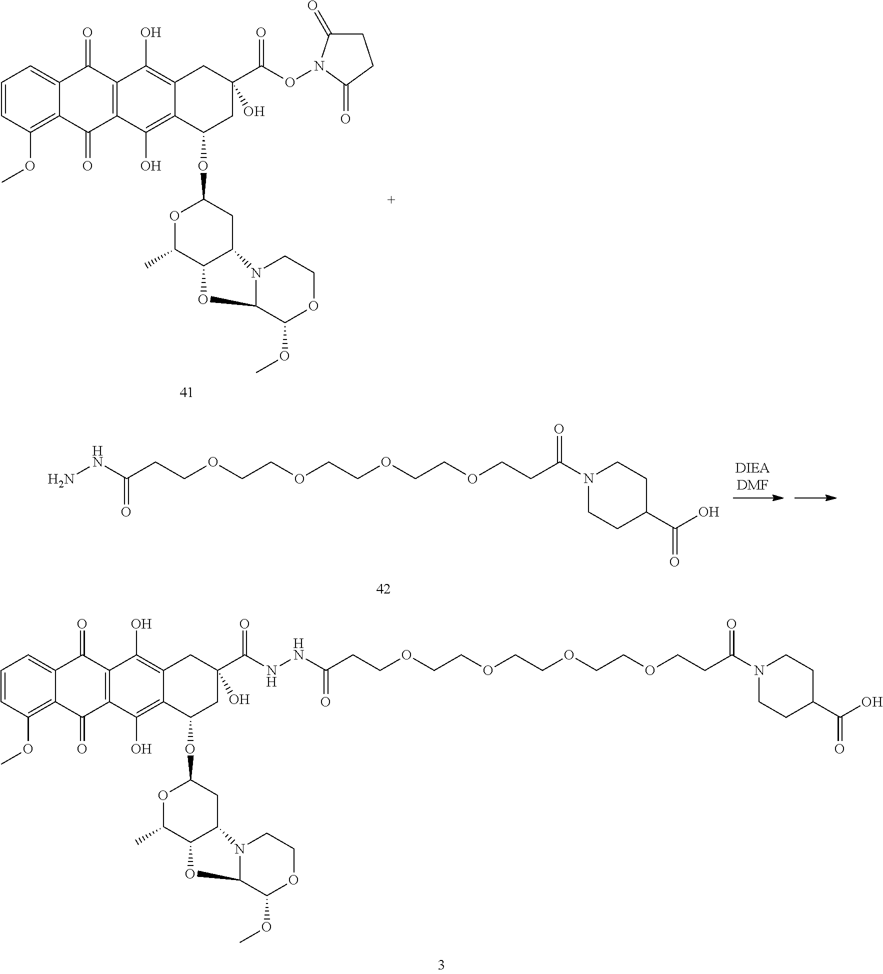 [SMILES: COc1cccc2c1C(=O)c1c(O)c3c(c(O)c1C2=O)C[C@@](O)(C(=O)ON1C(=O)CCC1=O)C[C@@H]3O[C@H]1C[C@H]2[C@H](O[C@@H]3[C@@H](OC)OCCN32)[C@H](C)O1 ; NNC(=O)CCOCCOCCOCCOCCC(=O)N1CCC(C(=O)O)CC1 ; COc1cccc2c1C(=O)c1c(O)c3c(c(O)c1C2=O)C[C@@](O)(C(=O)NNC(=O)CCOCCOCCOCCOCCC(=O)N1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and amine 42 (42 mg, 0.10 mmol). The mixture was stirred for 16 h, then evaporated and purified by column (silica gel, DCM:MeOH, 9:1) to give compound 3 (70 mg, 68%). MS m/z 1029.4 (M+H).

### Example 3. Preparation of Compound 4

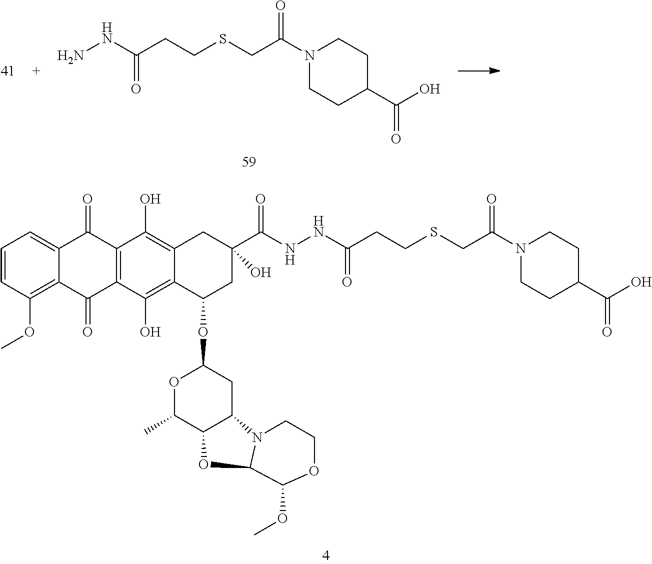 [SMILES: NNC(=O)CCSCC(=O)N1CCC(C(=O)O)CC1 ; COc1cccc2c1C(=O)c1c(O)c3c(c(O)c1C2=O)C[C@@](O)(C(=O)NNC(=O)CCSCC(=O)N1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and hydrazide 59 (43 mg, 0.15 mmol). The mixture was stirred at room temperature for 16 h, then diluted with DCM (30 mL). The mixture was washed with brine. The organic layer was dried and evaporated to dryness. The residue was purified by column (silica gel, DCM:MeOH, 9:1) to give compound 4 (56 mg, 62%). MS m/z 899.4 (M+H).

### Example 4. Preparation of Compound 5

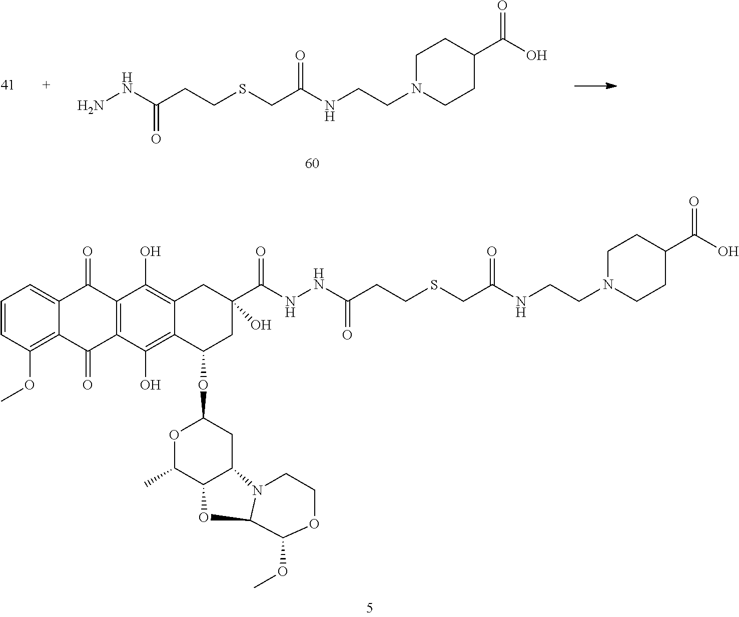 [SMILES: NNC(=O)CCSCC(=O)NCCN1CCC(C(=O)O)CC1 ; COc1cccc2c1C(=O)c1c(O)c3c(c(O)c1C2=O)C[C@@](O)(C(=O)NNC(=O)CCSCC(=O)NCCN1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and hydrazide 60 (50 mg, 0.15 mmol). The mixture was stirred at room temperature for 16 h, then diluted with DCM (30 mL). The mixture was washed with brine. The organic layer was dried and evaporated to dryness. The residue was purified by column (silica gel, DCM:MeOH, 9:1) to give compound 5 (41 mg, 44%). MS m/z 942.5 (M+H).

### Example 5. Preparation of Compound 6

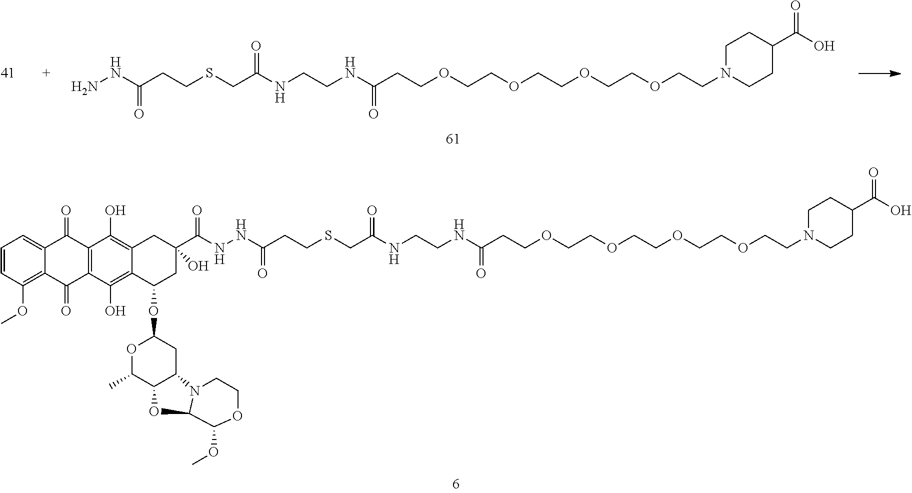 [SMILES: NNC(=O)CCSCC(=O)NCCNC(=O)CCOCCOCCOCCOCCN1CCC(C(=O)O)CC1 ; COc1cccc2c1C(=O)c1c(O)c3c(c(O)c1C2=O)C[C@@](O)(C(=O)NNC(=O)CCSCC(=O)NCCNC(=O)CCOCCOCCOCCOCCN1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and hydrazide 61 (87 mg, 0.15 mmol). The mixture was stirred at room temperature for 16 h, then diluted with DCM (50 mL). The mixture was washed with brine. The organic layer was dried and evaporated to dryness. The residue was purified by column (silica gel, DCM:MeOH, 9:1) to give compound 6 (47 mg, 40%). MS m/z 1186.5 (M+H).

### Example 6. Preparation of Compound 7

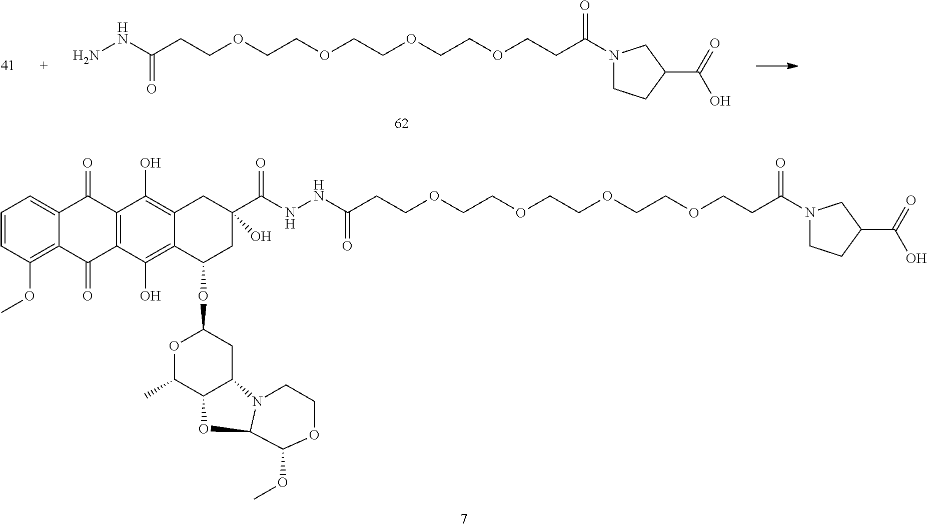 [SMILES: NNC(=O)CCOCCOCCOCCOCCC(=O)N1CCC(C(=O)O)C1 ; COc1cccc2c1C(=O)c1c(O)c3c(c(O)c1C2=O)C[C@@](O)(C(=O)NNC(=O)CCOCCOCCOCCOCCC(=O)N1CCC(C(=O)O)C1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (60 μL, 0.34 mmol), and hydrazide 62 (30 mg, 0.15 mmol). The mixture was stirred at room temperature for 16 h, then diluted with DCM (40 mL). The mixture was washed with brine. The organic layer was dried and evaporated to dryness. The residue was purified by column (silica gel, DCM:MeOH, 9:1) to give compound 7 (57 mg, 56%). MS m/z 1015.5 (M+H).

### Example 7. Preparation of Compound 8

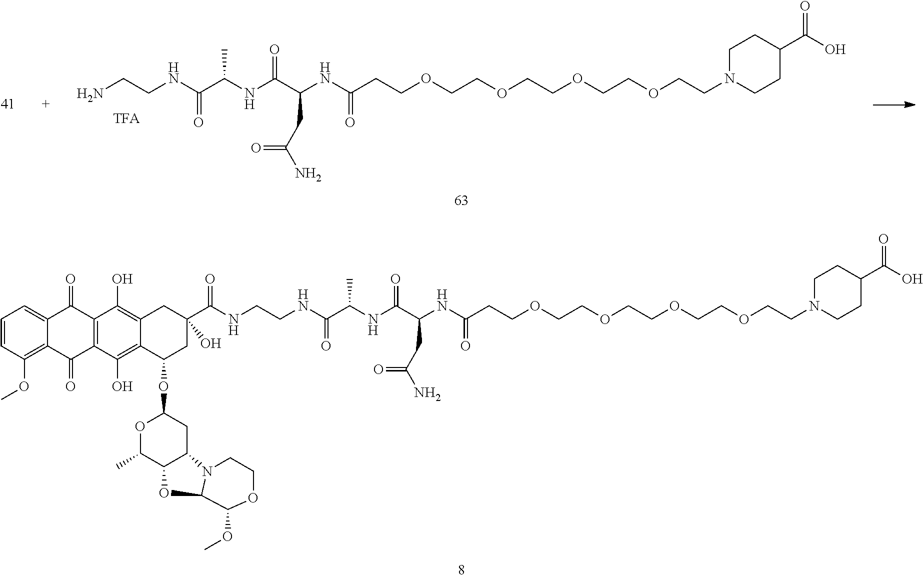 [SMILES: C[C@H](NC(=O)[C@H](CC(N)=O)NC(=O)CCOCCOCCOCCOCCN1CCC(C(=O)O)CC1)C(=O)NCCN ; COc1cccc2c1C(=O)c1c(O)c3c(c(O)c1C2=O)C[C@@](O)(C(=O)NCCNC(=O)[C@H](C)NC(=O)[C@H](CC(N)=O)NC(=O)CCOCCOCCOCCOCCN1CCC(C(=O)O)CC1)C[C@@H]3O[C@H]1C[C@H]2[C@H](O[C@@H]3[C@@H](OC)OCCN32)[C@H](C)O1]

To compound 41 (72 mg, 0.10 mmol) in 3 mL of DMF was added DIEA (75 μL), and amine. TFA 63 (86 mg, 0.12 mmol). The mixture was stirred at room temperature for 3 h, then diluted with DCM (40 mL). The mixture was washed with brine. The organic layer was dried and evaporated to dryness. The residue was purified by column (silica gel, DCM:MeOH, 9:1) to give compound 8 (63 mg, 52%). MS m/z 1214.5 (M+H).

### Example 8. Preparation of Compound 9

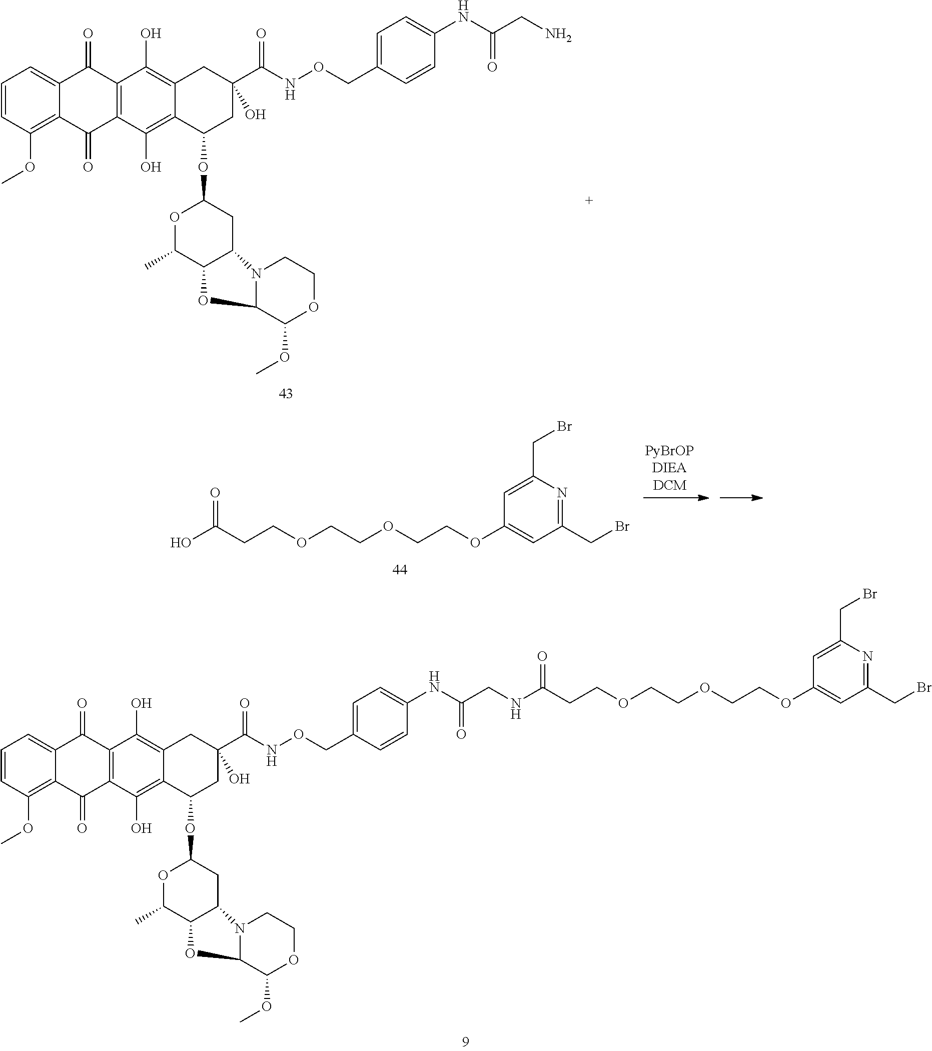 [SMILES: COc1cccc2c1C(=O)c1c(O)c3c(c(O)c1C2=O)C[C@@](O)(C(=O)NOCc1ccc(NC(=O)CN)cc1)C[C@@H]3O[C@H]1C[C@H]2[C@H](O[C@@H]3[C@@H](OC)OCCN32)[C@H](C)O1 ; O=C(O)CCOCCOCCOc1cc(CBr)nc(CBr)c1 ; COc1cccc2c1C(=O)c1c(O)c3c(c(O)c1C2=O)C[C@@](O)(C(=O)NOCc1ccc(NC(=O)CNC(=O)CCOCCOCCOc2cc(CBr)nc(CBr)c2)cc1)C[C@@H]3O[C@H]1C[C@H]2[C@H](O[C@@H]3[C@@H](OC)OCCN32)[C@H](C)O1]

To compound 44 (3.3 mg, 7.7 umol) in 2 mL of DMF was added DIEA (2.6 μL, 15 umol), PyBrOP (2.3 mg, 5 μmol), and amine 43 (2.5 mg, 3 μmol). The mixture was stirred for 10 min, then purified by column (silicagel, DCM:MeOH, 95:5) to give compound 9 (2.0 mg, 54%). MS m/z 1228.3 (M+H).

### Example 9. Preparation of Compound 10

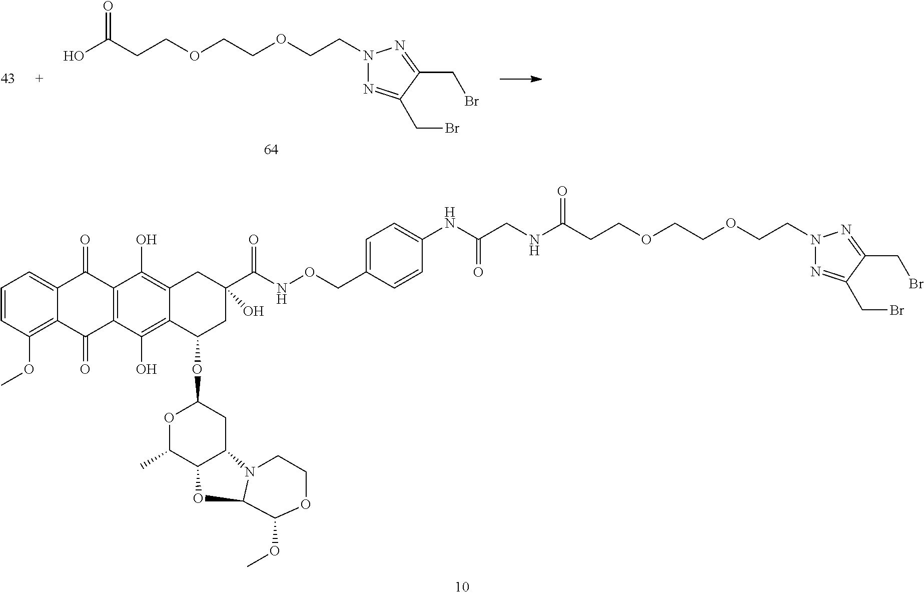 [SMILES: O=C(O)CCOCCOCCn1nc(CBr)c(CBr)n1 ; COc1cccc2c1C(=O)c1c(O)c3c(c(O)c1C2=O)C[C@@](O)(C(=O)NOCc1ccc(NC(=O)CNC(=O)CCOCCOCCn2nc(CBr)c(CBr)n2)cc1)C[C@@H]3O[C@H]1C[C@H]2[C@H](O[C@@H]3[C@@H](OC)OCCN32)[C@H](C)O1]

To compound 64 (10 mg, 23 umol) in 2 mL of DMF was added DIEA (8 μL, 50 umol), PyBrOP (7 mg, 15 μmol), and amine 43 (8 mg, 10 μmol). The mixture was stirred for 10 min, then purified by column (silicagel, DCM:MeOH, 90:10) to give compound 10 (5.0 mg, 42%). MS m/z 1202.3 (M+H).

### Example 10. Preparation of Compound 11

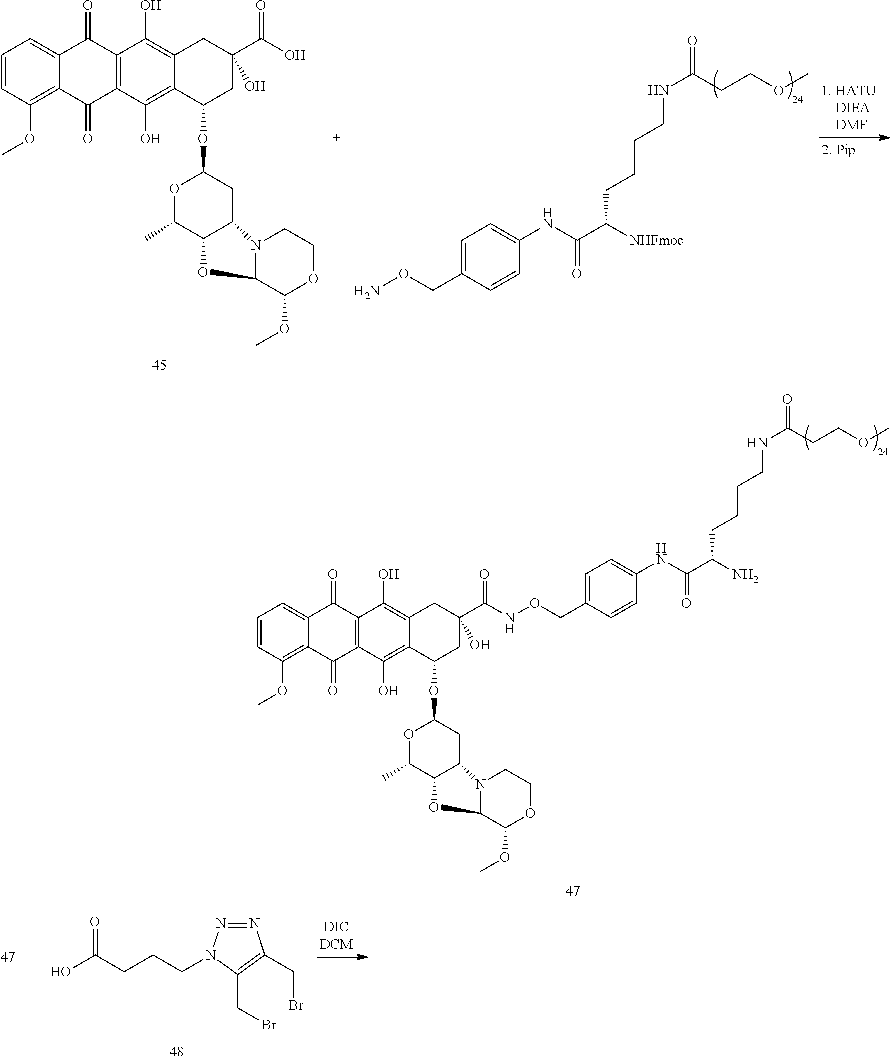

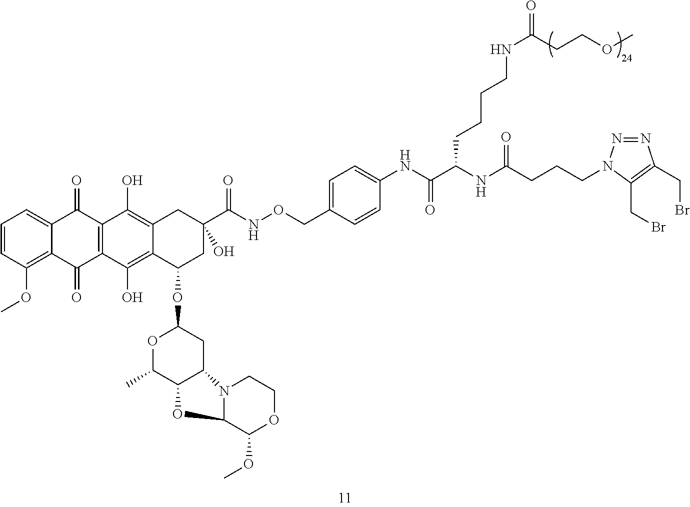

### Preparation of Compound 47

To compound 45 (17.7 mg, 28 μmol) in 2 mL of DMF was added DIEA (5 μL, 30 μmol), HATU (11 mg, 29 μmol), and amine 46 (48 mg, 28 μmol). The mixture was stirred for 30 min, then 100 μL of pipridine added. After 15 min, the mixture was evaporated and purified by HPLC to give compound 47 (18 mg, 30%). MS m/z 1974.7 (M+H).

### Preparation of Compound 11

To compound 48 (13.6 mg, 40 μmol) in 2 mL of DCM was added DIC (2.5 mg, 20 μmol), and amine 47 (18 mg, 9 μmol). The mixture was stirred for 30 min, then purified by HPLC to give compound 11 (9 mg, 43%). MS m/z 2296.8 (M+H).

### Example 11. Preparation of Compound 12

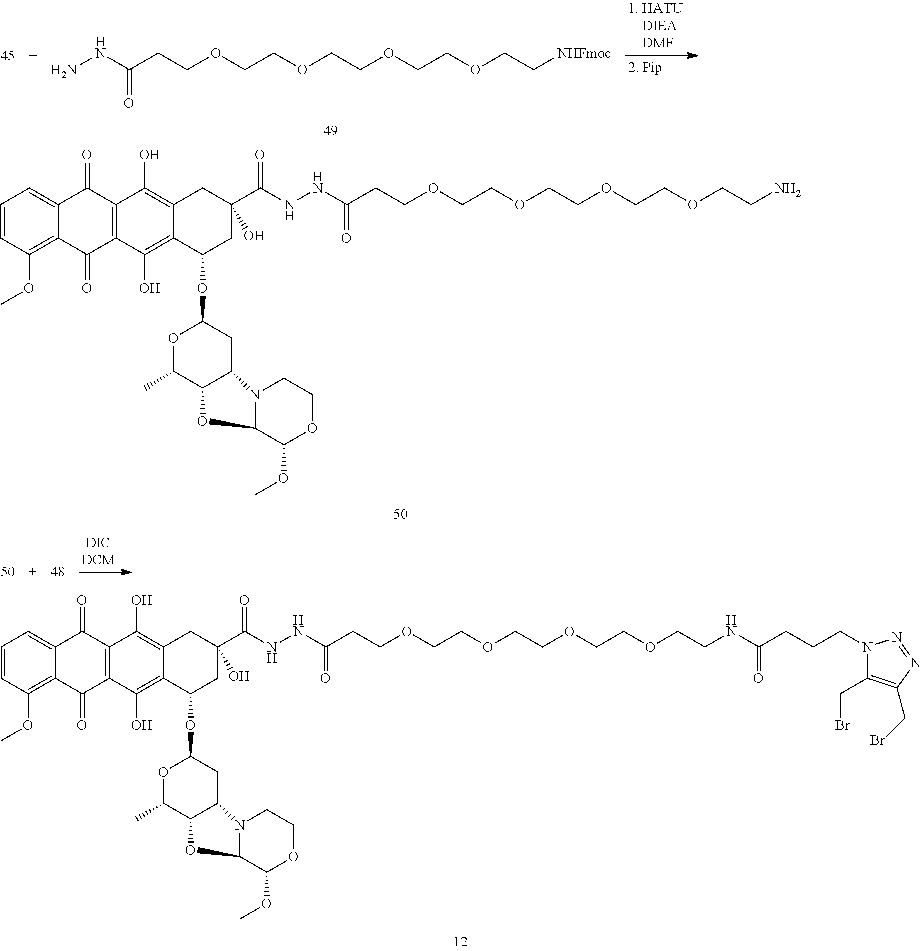

To compound 45 (45 mg, 72 μmol) in 2 mL of DMF was added DIEA (13 μL, 80 μmol), HATU (28 mg, 74 μmol), and amine 49 (36 mg, 72 μmol). The mixture was stirred for 30 min, then 100 μL of pipridine added. After 15 min, the mixture was evaporated and purified by HPLC to give compound 50 (16 mg, 25%). MS m/z 889.4 (M+H).

To compound 48 (13.6 mg, 40 μmol) in 2 mL of DCM was added DIC (2.5 mg, 20 μmol), and amine 50 (16 mg, 18 μmol). The mixture was stirred for 30 min, then purified by HPLC to give compound 12 (7 mg, 32%). MS m/z 1212.3 (M+H).

### Example 12. Preparation of Compound 13

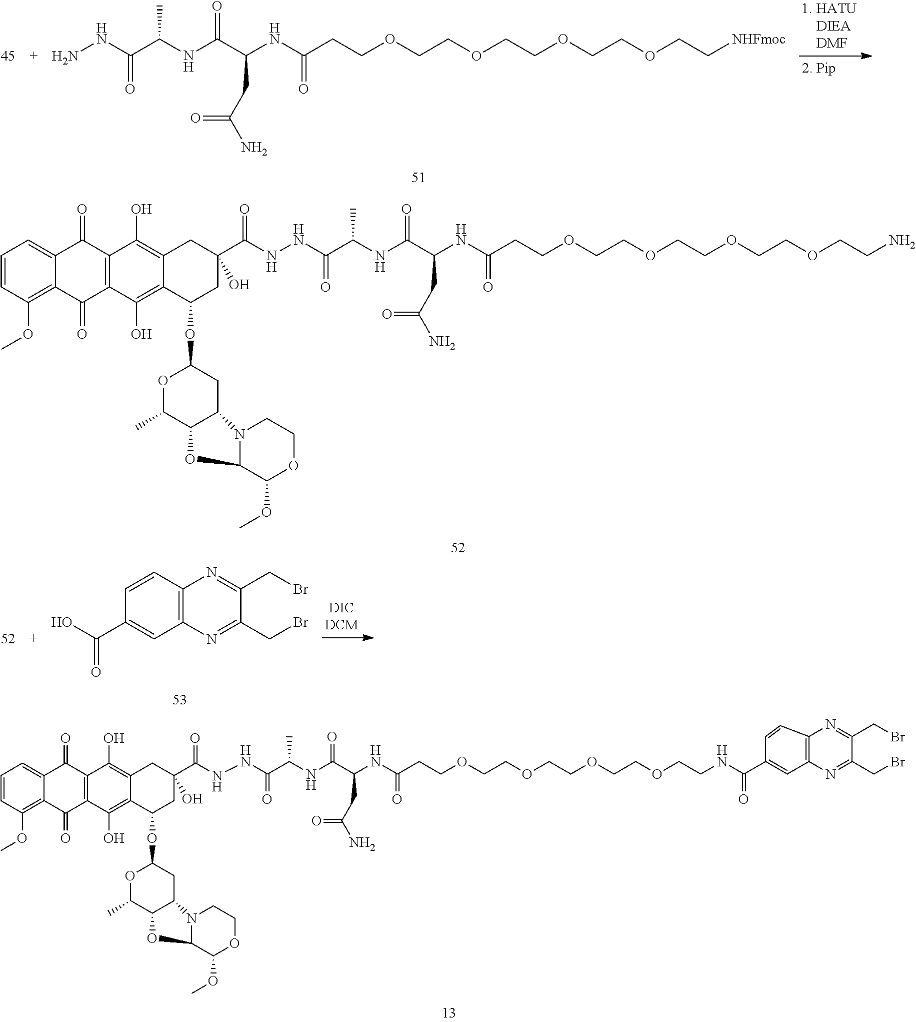

To compound 45 (45 mg, 72 μmol) in 2 mL of DMF was added DIEA (13 μL, 80 μmol), HATU (28 mg, 74 μmol), and amine 51 (49 mg, 72 μmol). The mixture was stirred for 30 min, then 100 μL of pipridine added. After 15 min, the mixture was evaporated and purified by HPLC to give compound 52 (27 mg, 35%). MS m/z 1074.4 (M+H).

To compound 53 (15 mg, 40 μmol) in 2 mL of DCM was added DIC (2.5 mg, 20 μmol), and amine 52 (21 mg, 20 μmol). The mixture was stirred for 30 min, then purified by HPLC to give compound 13 (13 mg, 47%). MS m/z 1416.3 (M+H).

### Example 13. Preparation of Compound 14

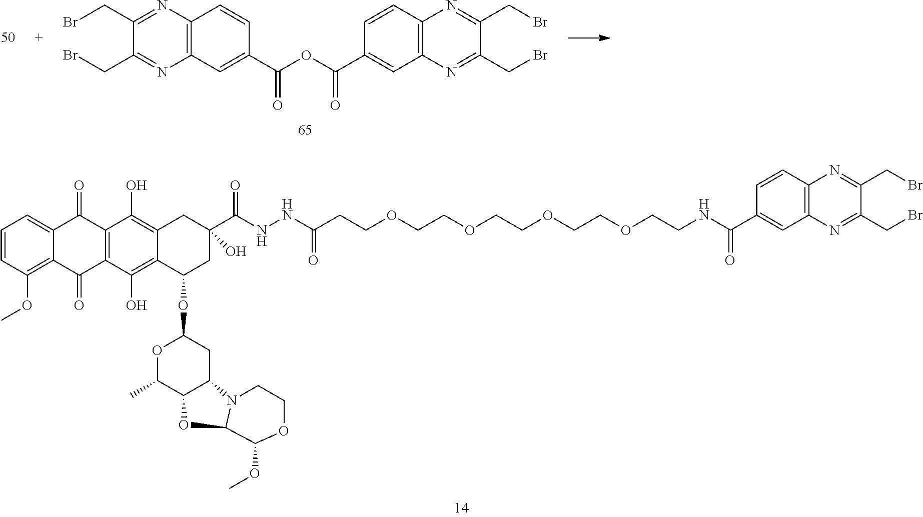 [SMILES: O=C(OC(=O)c1ccc2nc(CBr)c(CBr)nc2c1)c1ccc2nc(CBr)c(CBr)nc2c1 ; COc1cccc2c1C(=O)c1c(O)c3c(c(O)c1C2=O)C[C@@](O)(C(=O)NNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1]

To a solution of compound 50 (18 mg, 0.02 mmol) in DCM (2 mL) was added compound 65 (15 mg), followed by DIEA (5 μL). The mixture was stirred at room temperature for 10 min. The reaction was then diluted with DCM (30 mL) and washed with aq. saturated NaHCO₃. The organic layer was concentrated and residue was purified by RP-HPLC to give compound 14 as a red solid after lyophilization (7 mg, 29%). MS m/z 1231.3 (M+H).

### Example 14. Preparation of Compound 15

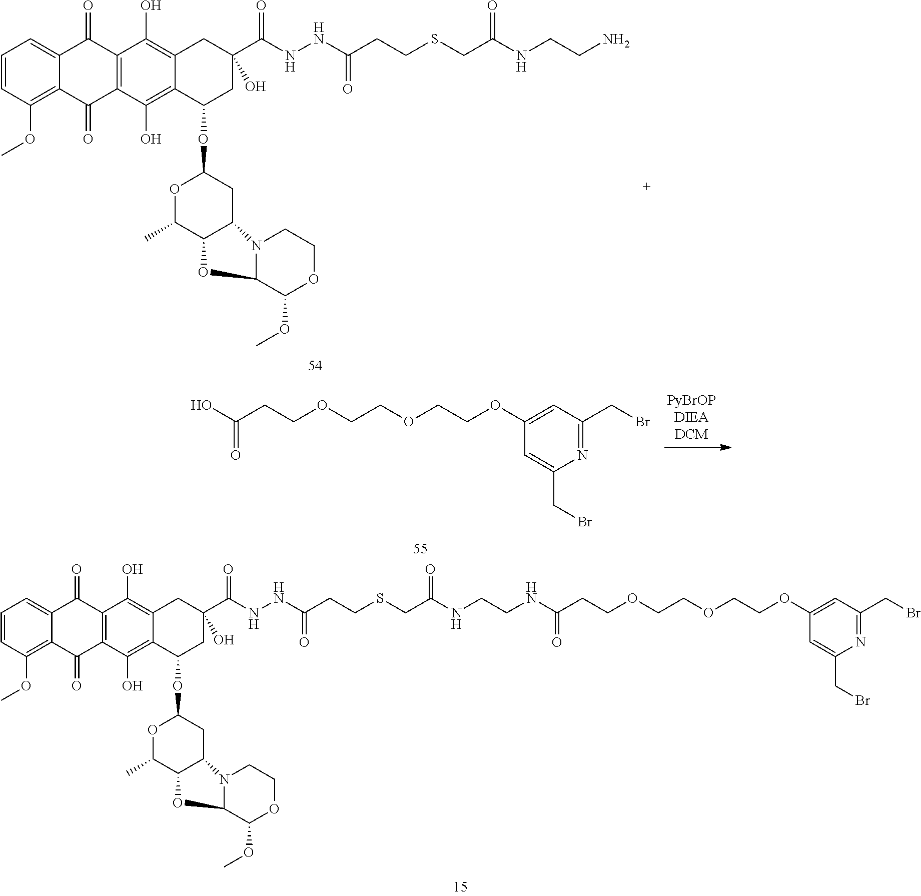 [SMILES: COc1cccc2c1C(=O)c1c(O)c3c(c(O)c1C2=O)C[C@@](O)(C(=O)NNC(=O)CCSCC(=O)NCCN)C[C@@H]3O[C@H]1C[C@H]2[C@H](O[C@@H]3[C@@H](OC)OCCN32)[C@H](C)O1 ; O=C(O)CCOCCOCCOc1cc(CBr)nc(CBr)c1 ; COc1cccc2c1C(=O)c1c(O)c3c(c(O)c1C2=O)C[C@@](O)(C(=O)NNC(=O)CCSCC(=O)NCCNC(=O)CCOCCOCCOc1cc(CBr)nc(CBr)c1)C[C@@H]3O[C@H]1C[C@H]2[C@H](O[C@@H]3[C@@H](OC)OCCN32)[C@H](C)O1]

To compound 55 (9 mg, 20 μmol) in 2 mL of DCM was added PyBrOP (9 mg, 20 μmol), DIEA (8 μL, 80 μmol), and amine 54 (15 mg, 20 μmol). The mixture was stirred for 30 min, then evaporated and purified by HPLC to give compound 15 (9 mg, 37%). MS m/z 1253.2 (M+H).

### Example 15. Preparation of Compound 16

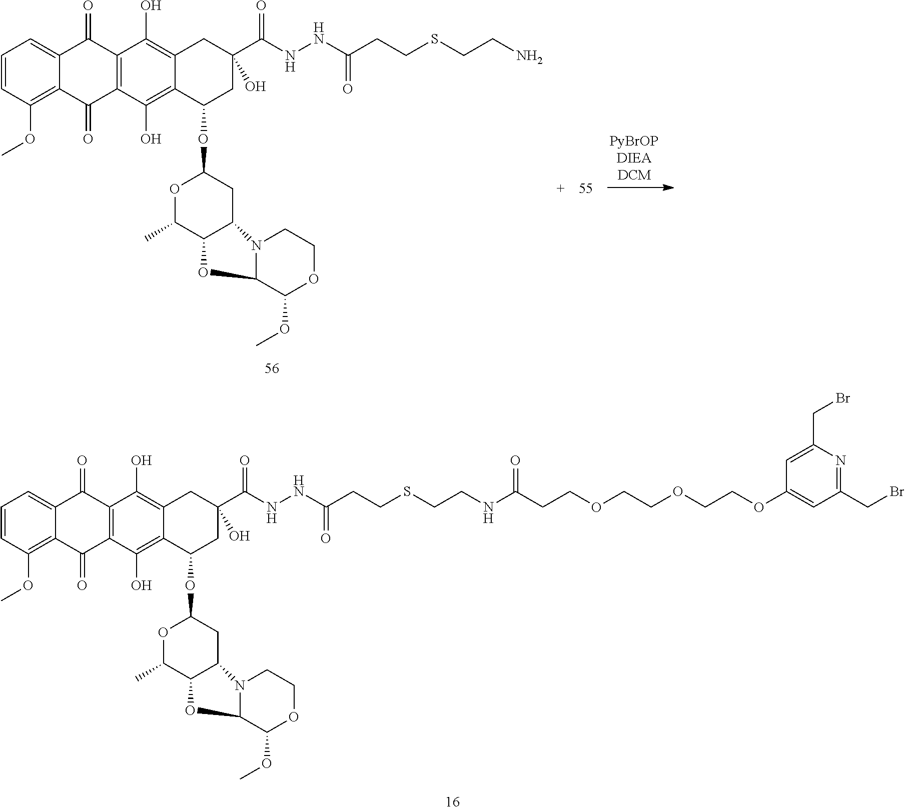 [SMILES: COc1cccc2c1C(=O)c1c(O)c3c(c(O)c1C2=O)C[C@@](O)(C(=O)NNC(=O)CCSCCN)C[C@@H]3O[C@H]1C[C@H]2[C@H](O[C@@H]3[C@@H](OC)OCCN32)[C@H](C)O1 ; COc1cccc2c1C(=O)c1c(O)c3c(c(O)c1C2=O)C[C@@](O)(C(=O)NNC(=O)CCSCCNC(=O)CCOCCOCCOc1cc(CBr)nc(CBr)c1)C[C@@H]3O[C@H]1C[C@H]2[C@H](O[C@@H]3[C@@H](OC)OCCN32)[C@H](C)O1]

To compound 55 (9 mg, 20 μmol) in 2 mL of DCM was added PyBrOP (9 mg, 20 μmol), DIEA (8 μL, 80 μmol), and amine 56 (15 mg, 20 μmol). The mixture was stirred for 30 min, then evaporated and purified by HPLC to give compound 16 (8 mg, 33%). MS m/z 1196.2 (M+H).

### Example 16. Preparation of Compound 17

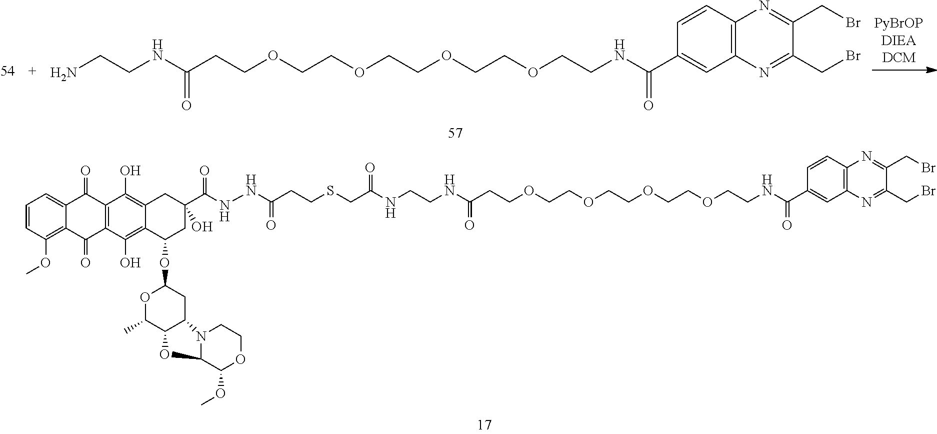 [SMILES: NCCNC(=O)CCOCCOCCOCCOCCNC(=O)c1ccc2nc(CBr)c(CBr)nc2c1 ; COc1cccc2c1C(=O)c1c(O)c3c(c(O)c1C2=O)C[C@@](O)(C(=O)NNC(=O)CCSCC(=O)NCCNC(=O)CCOCCOCCOCCOCCNC(=O)c1ccc2nc(CBr)c(CBr)nc2c1)C[C@@H]3O[C@H]1C[C@H]2[C@H](O[C@@H]3[C@@H](OC)OCCN32)[C@H](C)O1]

To compound 57 (12 mg, 20 μmol) in 2 mL of DCM was added PyBrOP (9 mg, 20 μmol), DIEA (8 μL, 80 μmol), and amine 54 (15 mg, 20 μmol). The mixture was stirred for 30 min, then evaporated and purified by HPLC to give compound 17 (13 mg, 47%). MS m/z 1419.3 (M+H).

### Example 17. Preparation of Compound 18

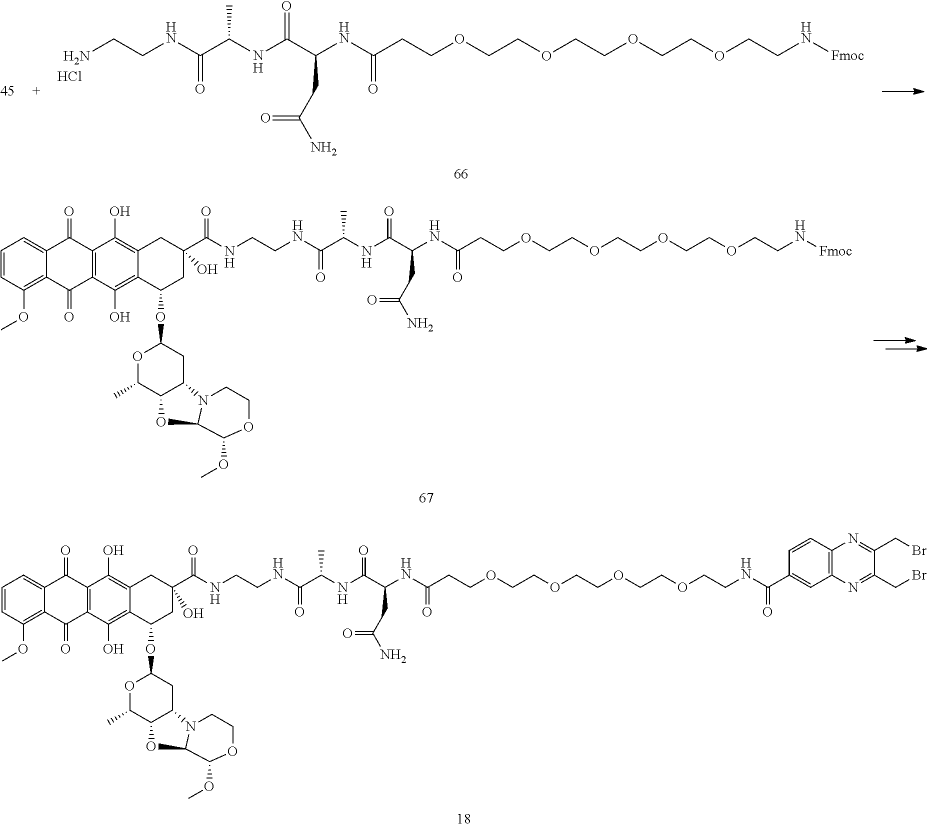

To a solution of compound 45 (63 mg, 0.1 mmol) in DMF (3 mL) was added compound 66 (75 mg, 0.1 mmol), followed by DIEA (70 μL) and HATU (40 mg). The mixture was stirred at room temperature for 5 min, then diluted with DCM (50 mL). The mixture was washed with aq. saturated NaHCO₃ and brine. The organic layer was dried and concentrated. The crude product was purified by column chromatography (silica gel, MeOH/DCM: 1/19, /v/v) to give compound 67 as a red solid (81 mg, 61%)

Compound 67 (66 mg, 0.05 mmol) was dissolved in DMF (2 mL). Pipridine (100 μL) was added. The mixture was stirred at room temperature for 30 min and then concentrated to dryness under reduced pressure. The residue was redissolved in DCM (3 mL). Anhydride 65 (42 mg) was added, followed by DIEA (18 μL). After 30 min, the reaction was concentrated and the crude product was purified by RP-HPLC to give compound 18 as a red solid (52 mg, 72%). MS m/z 1444.5 (M+H).

### Example 18

This example provides the results of EC50 assays of the designated drug conjugated antibodies measured in vitro in specified cells. ADC 70 was synthesized from an unmodified PNU-159682 (WO 2010/009124 A2) conjugated to an anti-Her 2 antibody as a comparison. Most of ADCs disclosed here showed much improved safety characteristics (ADC 21-29, 31, and 35) and some ADCs showed improved cell king efficacy (ADC 26, 30, 31, and 34).

| | | | | | | MDA- | MDA- | MDA- |
|---|---|---|---|---|---|---|---|---|
| | SBKR3 | HCC1954 | MCF7 | MDAMB | BT474 | MB-453 | MB-175 | MB-361 |
| Conjugate | (Her2+++) | (Her2+++) | (Her2+) | 468 (−) | (Her2+++) | (Her2++) | (Her2+) | (Her2+++) |
| ID | EC50 [nM] | | | | | | | |
| 20 | 0.030 | 0.400 | 11.660 | 5.648 | | | | |
| 21 | 0.072 | | | 29.130 | 0.885 | | 0.740 | |
| 22 | 0.074 | | | 13.570 | 0.778 | 9.566 | 0.697 | |
| 26 | 0.022 | | | 14.370 | 0.374 | 0.403 | | 0.124 |
| 28 | 0.031 | 0.541 | ~80 | 14.910 | | ~30 | 11.820 | |
| 29 | 0.049 | 0.343 | ~100 | 31.480 | | ~50 | ~20 | |
| 30 | 0.023 | 0.066 | 18.740 | 3.393 | 0.143 | 0.169 | 0.264 | 0.063 |
| 31 | 0.028 | 0.128 | | 12.560 | 0.283 | 0.270 | | 0.087 |
| 32 | 0.034 | | | 0.775 | 0.108 | 0.210 | 0.057 | 0.093 |
| 34 | 0.011 | 0.144 | 1.619 | 0.923 | | 0.167 | 0.655 | |
| 35 | 0.062 | 0.111 | | 59.180 | 0.296 | 0.194 | 0.137 | 0.057 |
| 70 | 0.033 | | 10 | 5 | | | | |

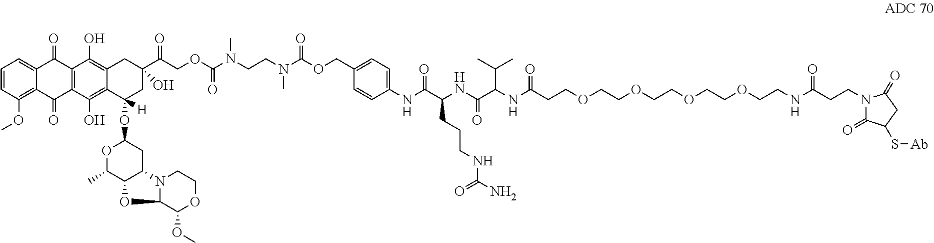

### Example 19

This example shows in vivo efficacy of ADC 20 (an anti-Her2 antibody conjugate) in a Subcutaneous N87 Xenograft Model. FIG. 1 shows a single dose of conjugate 20 administered to BALB/c nude mice by intravenous administration. There were 8 mice in each group and total 3 groups of mice were studied: 1 group of mice was injected with T-DM1 (Trastuzumab-DM1 conjugate); one group of mice was injected with ADC 20; and one vehicle control. All the drugs were administered in the same manner (single dose). A single dose of ADC-20 iv. at 1 mg/kg outperformed T-DM1 at 2 mg/kg and completely inhibited tumor growth up to 58 days.

### Example 20

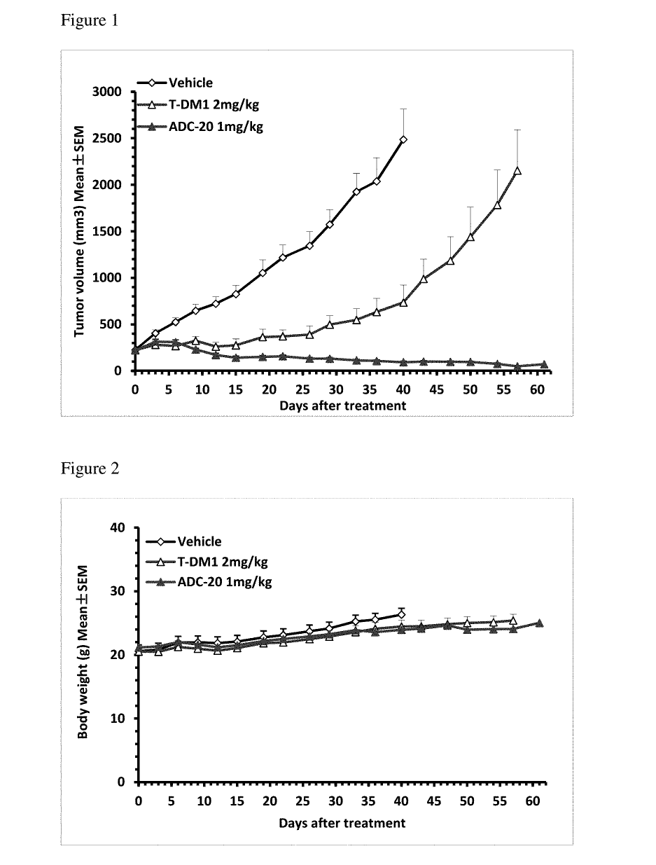

This example shows in vivo safety of ADC 20 (an anti-Her2 antibody conjugate) in a Subcutaneous N87 Xenograft Model. FIG. 2 shows a single dose of conjugate 20 administered to BALB/c nude mice by intravenous administration. There were 8 mice in each group and total 3 groups of mice were studied: 1 group of mice was injected with T-DM1 (Trastuzumab-DM1 conjugate); 1 group of mice was injected with ADC 20; and one vehicle control. All the drugs were administered in the same manner (single dose). A single dose of ADC-20 iv. at 1 mg/kg did not retard body weight gain and was comparable to that of T-DM1

### Example 21

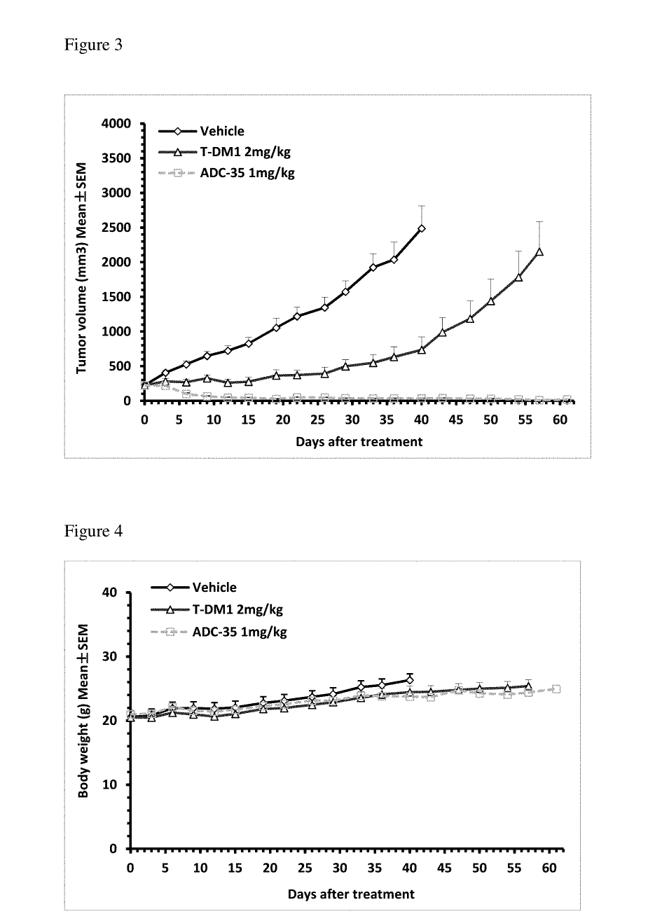

This example shows in vivo efficacy of ADC 35 (an anti-Her2 antibody conjugate) in a Subcutaneous N87 Xenograft Model. FIG. 3 shows a single dose of conjugate 30 administered to BALB/c nude mice by intravenous administration. There were 8 mice in each group and total 3 groups of mice were studied: 1 group of mice was injected with T-DM1 (Trastuzumab-DM1 conjugate); 1 group of mice was injected with ADC 20; and one vehicle control. All the drugs were administered in the same manner (single dose). A single dose of ADC-35 iv. at 1 mg/kg outperformed T-DM1 at 2 mg/kg and completely inhibited tumor growth up to 58 days.

### Example 22

This example shows in vivo safety of ADC 35 (an anti-Her2 antibody conjugate) in a Subcutaneous N87 Xenograft Model. FIG. 4 shows a single dose of conjugate 30 administered to BALB/c nude mice by intravenous administration. There were 8 mice in each group and total 3 groups of mice were studied: 1 group of mice was injected with T-DM1 (Trastuzumab-DM1 conjugate); 1 group of mice was injected with ADC 20; and one vehicle control. All the drugs were administered in the same manner (single dose). A single dose of ADC-35 iv. at 1 mg/kg did not retard body weight gain and was comparable to that of T-DM1.

### Example 23

This example shows a general conjugation procedure for synthesizing antibody drug conjugates 19, 20, 21, 22, 23, 24 and 25 (Table 3 above). To a solution of 0.5-50 mg/mL of antibody in buffer at pH 6.0-9.0 with 0-30% organic solvent, was added 0.1-10 eq of activated drug linker conjugate (2, or 3, or 4, or 5, or 6, or 7, or 8) in a manner of portion wise or continuous flow. The reaction was performed at 0-40° C. for 0.5-50 hours with gentle stirring or shaking, monitored by HIC-HPLC. The resultant crude ADC product underwent necessary down-stream steps of desalt, buffet changes/formulation, and optionally, purification, using the state-of-art procedures. The ADC product was characterized by HIC-HPLC, SEC, RP-HPLC, and optionally LC-MS.

### Example 24

This example shows the general conjugation procedure for synthesizing antibody drug conjugates 26, 27, 28, 29, 30, 31, 32, 33, 34 and 35 (Table 3 above). To a solution of antibody, 0.5-50 mgs/mL, in a certain buffet at pH 5.0-9.0, such as PBS, was added 0.5-100 eq of reducing agent such as TCEP and DTT. The reduction was performed at 0-40° C. for 0.5-40 hours with gentle stirring or shaking, and then the reducing agent was removed by column or ultrafiltration. To the reduced antibody, 0.5-50 mg/mL, in a certain buffet at pH 5.0-9.0, such as PBS, with 0-30% of organic co-solvent such as DMA, was added 0.5-10 eq of the drug-linker reactant (selected from compound 9). The reaction was conducted at 0-40° C. for 0.5-40 hours with gentle stirring or shaking, monitored by HIC-HPLC. The resultant crude ADC product underwent necessary down-stream steps of desalt, buffet changes/formulation, and optionally, purification, using the state-of-art procedures. The final ADC product was characterized by HIC-HPLC, SEC, RP-HPLC, and optionally LC-MS.

We claim:

1. An antibody drug conjugate (ADC) having a structure of Formula I
AbL¹-L²-D)ₙ (I)
or a pharmaceutically acceptable salt thereof,
wherein:
Ab is an antibody;
L¹ is a connector;
L² is a linker selected from the group consisting of an amino acid, a peptide, —(CH₂)ₙ—, —(CH₂CH₂O)ₙ—, PAB, Val-Cit-PAB, Val-Ala-PAB, Ala-Ala-Asn-PAB, and combinations thereof;
wherein -L¹-L² is selected from the group consisting of 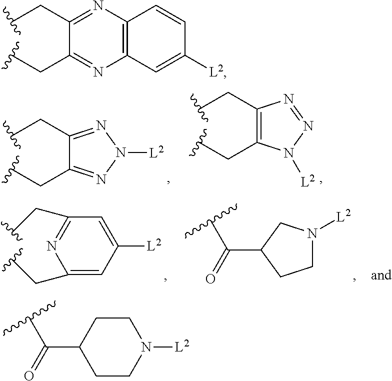
D is a drug moiety having a structure of Formula II 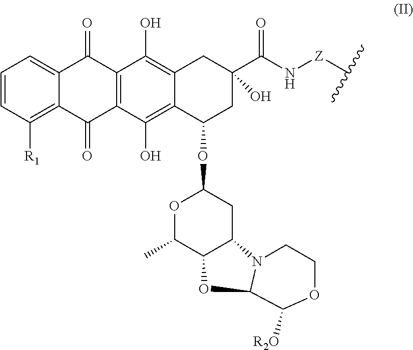
wherein Z═O, NH or CH₂;
R₁═H, OH, or OMe; and
R₂ is a C1-C5 alkyl group.
n is an integer from 1-10.

2. The ADC of claim 1, wherein Formula I is a composition selected from the group consisting of 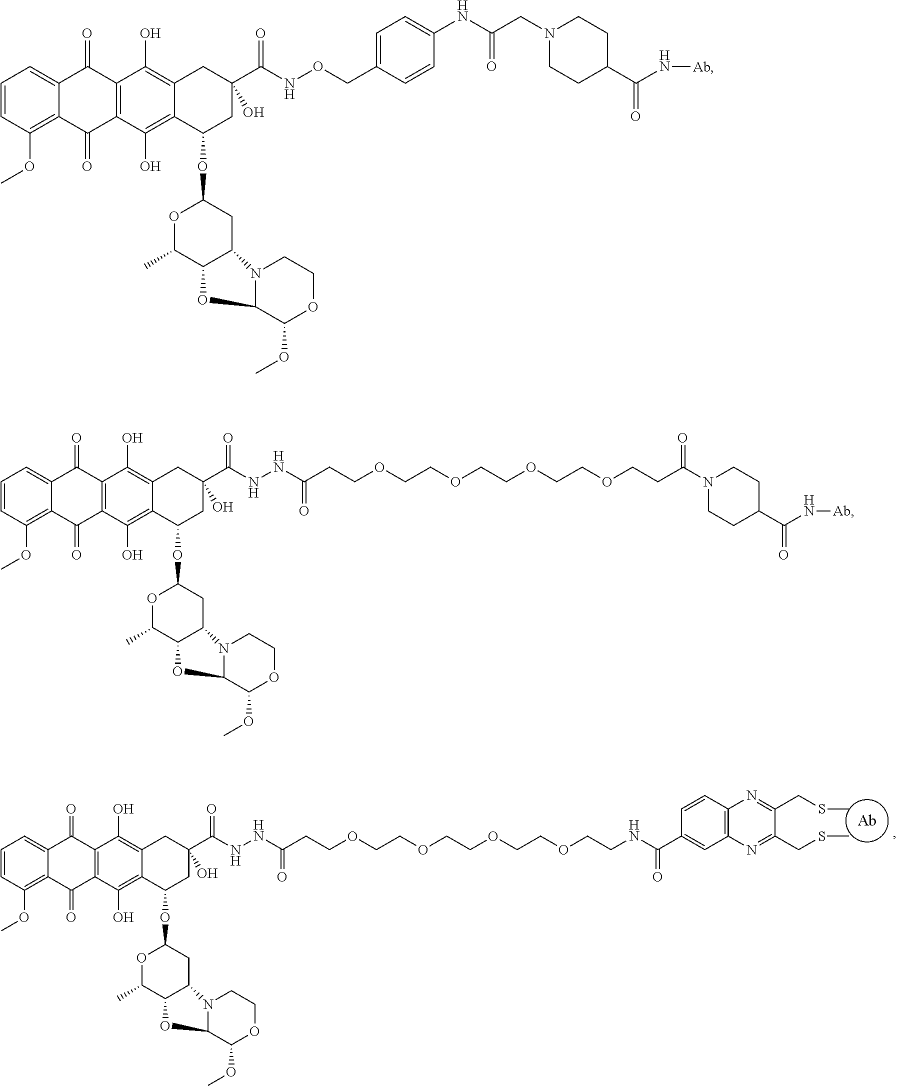 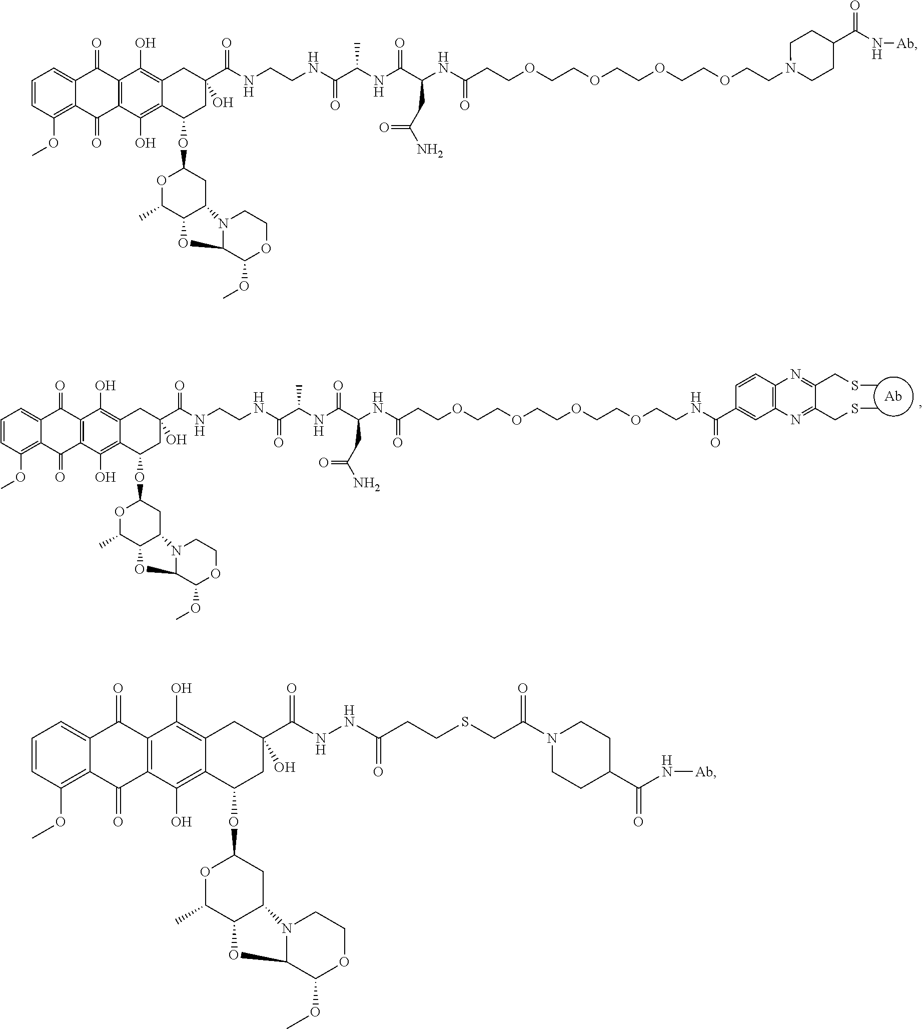 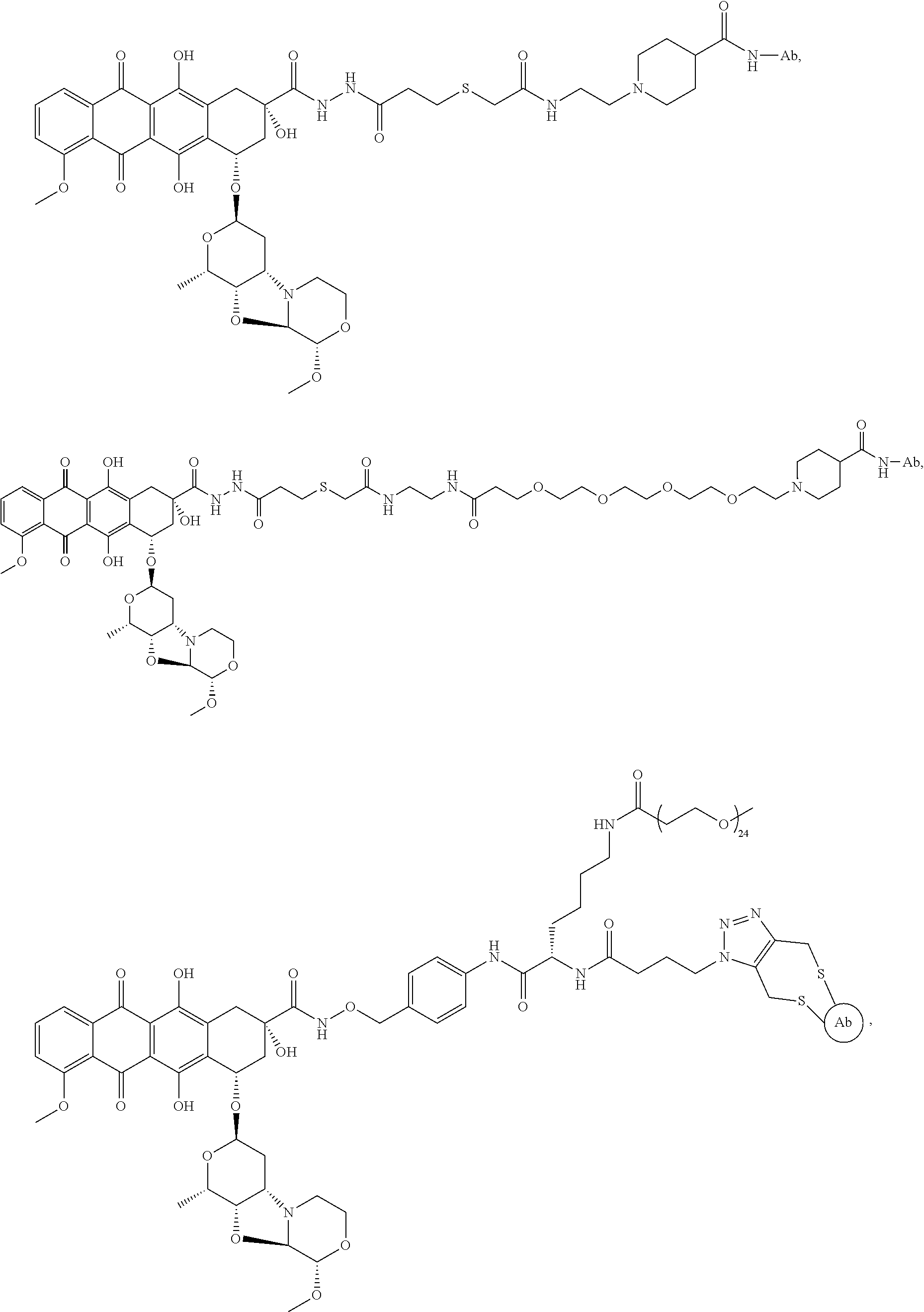 [chemical structure]
